SEQUENCE LISTING (16 sequences)

SEQ ID NO: 1 - RNA, 21 nt, synthetic construct
  modified_base 20..21: mod_base = OTHER; note = thymine
  misc_feature 1..21: note = Synthetic: siFLuc sense
  source 1..21: mol_type = other RNA
tcgaagtactcagcgtaagtt

SEQ ID NO: 2 - RNA, 21 nt, synthetic construct
  modified_base 20..21: mod_base = OTHER; note = thymine
  misc_feature 1..21: note = Synthetic: siFLuc antisense
  source 1..21: mol_type = other RNA
cttacgctgagtacttcgatt

SEQ ID NO: 3 - RNA, 25 nt, synthetic construct
  misc_feature 1..25: note = Synthetic: siFLuc-2 sense
  modified_base 1: mod_base = OTHER; note = 2 -O-methyl RNA base
  modified_base 2: mod_base = OTHER; note = 2 -O-methyl RNA base
  modified_base 4: mod_base = OTHER; note = 2 -O-methyl RNA base
  modified_base 6: mod_base = OTHER; note = 2 -O-methyl RNA base
  modified_base 12: mod_base = OTHER; note = 2 -O-methyl RNA base
  modified_base 14: mod_base = OTHER; note = 2 -O-methyl RNA base
  modified_base 16: mod_base = OTHER; note = 2 -O-methyl RNA base
  modified_base 18: mod_base = OTHER; note = 2 -O-methyl RNA base
  modified_base 24: mod_base = OTHER; note = 2 -O-methyl RNA base
  modified_base 25: mod_base = OTHER; note = deoxyadenosine
  source 1..25: mol_type = other RNA
ggttcctggaacaattgcttttaca

SEQ ID NO: 4 - RNA, 27 nt, synthetic construct
  misc_feature 1..27: note = Synthetic: siFLuc-2 antisense
  modified_base 3: mod_base = OTHER; note = 2 -O-methyl RNA base
  modified_base 9: mod_base = OTHER; note = 2 -O-methyl RNA base
  modified_base 11: mod_base = OTHER; note = 2 -O-methyl RNA base
  modified_base 13: mod_base = OTHER; note = 2 -O-methyl RNA base
  modified_base 23: mod_base = OTHER; note = 2 -O-methyl RNA base
  modified_base 25: mod_base = OTHER; note = 2 -O-methyl RNA base
  modified_base 26: mod_base = OTHER; note = 2 -O-methyl RNA base
  modified_base 27: mod_base = OTHER; note = 2 -O-methyl RNA base
  source 1..27: mol_type = other RNA
tgtaaaagcaattgttccaggaaccag

SEQ ID NO: 5 - RNA, 21 nt, synthetic construct
  modified_base 20..21: mod_base = OTHER; note = thymine
  misc_feature 1..21: note = Synthetic: sinLuc sense
  source 1..21: mol_type = other RNA
ggattgtcctgagcggtgatt

SEQ ID NO: 6 - RNA, 21 nt, synthetic construct
  modified_base 20..21: mod_base = OTHER; note = thymine
  misc_feature 1..21: note = Synthetic: sinLuc antisense
  source 1..21: mol_type = other RNA
tcaccgctcaggacaatcctt

SEQ ID NO: 7 - RNA, 25 nt, synthetic construct
  modified_base 25: mod_base = OTHER; note = thymine
  misc_feature 1..25: note = Synthetic: siCCNE1 sense
  modified_base 1: mod_base = OTHER; note = 2 -O-methyl RNA base
  modified_base 2: mod_base = OTHER; note = 2 -O-methyl RNA base
  modified_base 4: mod_base = OTHER; note = 2 -O-methyl RNA base
  modified_base 6: mod_base = OTHER; note = 2 -O-methyl RNA base
  modified_base 12: mod_base = OTHER; note = 2 -O-methyl RNA base
  modified_base 14: mod_base = OTHER; note = 2 -O-methyl RNA base
  modified_base 16: mod_base = OTHER; note = 2 -O-methyl RNA base
  modified_base 18: mod_base = OTHER; note = 2 -O-methyl RNA base
  modified_base 24: mod_base = OTHER; note = 2 -O-methyl RNA base
  source 1..25: mol_type = other RNA
gcttcggccttgtatcatttctcgt

SEQ ID NO: 8 - RNA, 27 nt, synthetic construct
  misc_feature 1..27: note = Synthetic: siCCNE1antisense
  modified_base 3: mod_base = OTHER; note = 2 -O-methyl RNA base
  modified_base 9: mod_base = OTHER; note = 2 -O-methyl RNA base
  modified_base 11: mod_base = OTHER; note = 2 -O-methyl RNA base
  modified_base 13: mod_base = OTHER; note = 2 -O-methyl RNA base
  modified_base 23: mod_base = OTHER; note = 2 -O-methyl RNA base
  modified_base 25: mod_base = OTHER; note = 2 -O-methyl RNA base
  modified_base 26: mod_base = OTHER; note = 2 -O-methyl RNA base
  modified_base 27: mod_base = OTHER; note = 2 -O-methyl RNA base
  source 1..27: mol_type = other RNA
acgagaaatgatacaaggccgaagctg

SEQ ID NO: 9 - RNA, 25 nt, synthetic construct
  misc_feature 1..25: note = Synthetic: siNT sense
  modified_base 1: mod_base = OTHER; note = 2 -O-methyl RNA base
  modified_base 2: mod_base = OTHER; note = 2 -O-methyl RNA base
  modified_base 4: mod_base = OTHER; note = 2 -O-methyl RNA base
  modified_base 6: mod_base = OTHER; note = 2 -O-methyl RNA base
  modified_base 12: mod_base = OTHER; note = 2 -O-methyl RNA base
  modified_base 14: mod_base = OTHER; note = 2 -O-methyl RNA base
  modified_base 16: mod_base = OTHER; note = 2 -O-methyl RNA base
  modified_base 18: mod_base = OTHER; note = 2 -O-methyl RNA base
  modified_base 24: mod_base = OTHER; note = 2 -O-methyl RNA base
  modified_base 25: mod_base = OTHER; note = deoxycytosine
  source 1..25: mol_type = other RNA
ataccttcccaggtaacaaaccaac

SEQ ID NO: 10 - RNA, 27 nt, synthetic construct
  misc_feature 1..27: note = Synthetic: siNT antisense
  modified_base 3: mod_base = OTHER; note = 2 -O-methyl RNA base
  modified_base 9: mod_base = OTHER; note = 2 -O-methyl RNA base
  modified_base 11: mod_base = OTHER; note = 2 -O-methyl RNA base
  modified_base 13: mod_base = OTHER; note = 2 -O-methyl RNA base
  modified_base 23: mod_base = OTHER; note = 2 -O-methyl RNA base
  modified_base 25: mod_base = OTHER; note = 2 -O-methyl RNA base
  modified_base 26: mod_base = OTHER; note = 2 -O-methyl RNA base
  modified_base 27: mod_base = OTHER; note = 2 -O-methyl RNA base
  source 1..27: mol_type = other RNA
gttggtttgttacctgggaaggtataa

SEQ ID NO: 11 - DNA, 22 bp, synthetic construct
  misc_feature 1..22: note = Synthetic: CCNE118 Forward
  source 1..22: mol_type = other DNA
tgtgtcctggatgttgactgcc

SEQ ID NO: 12 - DNA, 23 bp, synthetic construct
  misc_feature 1..23: note = Synthetic: CCNE118 Reverse
  source 1..23: mol_type = other DNA
ctctatgtcgcaccactgatacc

SEQ ID NO: 13 - DNA, 24 bp, synthetic construct
  misc_feature 1..24: note = Synthetic: GREB119 Forward
  source 1..24: mol_type = other DNA
caaagaataacctgttggccctgc

SEQ ID NO: 14 - DNA, 24 bp, synthetic construct
  misc_feature 1..24: note = Synthetic: GREB119 Reverse
  source 1..24: mol_type = other DNA
gacatgcctgcgctctcatactta

SEQ ID NO: 15 - DNA, 30 bp, synthetic construct
  misc_feature 1..30: note = Synthetic: ACTB20 Forward
  source 1..30: mol_type = other DNA
ccaaccgcgagaagatgacccagatcatgt

SEQ ID NO: 16 - DNA, 30 bp, synthetic construct
  misc_feature 1..30: note = Synthetic: ACTB20 Reverse
  source 1..30: mol_type = other DNA
gtgaggatcttcatgaggtagtcagtcagg

(19) United States
(12) Patent Application Publication
(10) Pub. No.: US 2025/0017856 A1
(43) Pub. Date: Jan. 16, 2025

(54) CO-FORMULATION AND CO-DELIVERY OF IONIZABLE COLLOID FORMING DRUGS WITH NUCLEIC ACIDS

(71) Applicant: THE GOVERNING COUNCIL OF THE UNIVERSITY OF TORONTO, Toronto (CA)

(72) Inventors: Kai Vincent Slaughter, York (CA); Molly Sandra Shoichet, Toronto (CA); Eric Niel Rummens Donders, Etobicoke (CA)

(73) Assignee: THE GOVERNING COUNCIL OF THE UNIVERSITY OF TORONTO, Toronto (CA)

(21) Appl. No.: 18/773,063

(22) Filed: Jul. 15, 2024

Related U.S. Application Data
(60) Provisional application No. 63/526,991, filed on Jul. 15, 2023.

(51) Int. Cl. A61K 9/127; A61K 9/51; A61K 31/4375; A61K 31/44; A61K 31/565; A61K 31/7088; A61K 45/06

(52) U.S. Cl. CPC A61K 9/1271; A61K 9/1277; A61K 9/5123; A61K 31/4375; A61K 31/44; A61K 31/565; A61K 31/7088; A61K 45/06

(57) ABSTRACT

A co-formulation comprising: (i) an ionizable colloid forming active substance; (ii) a nucleic acid molecule; and (iii) an amphiphilic molecule. Also, a method of treating a disease or disorder comprising administering the co-formulation to a subject in need, wherein the ionizable colloid-forming active substance and the nucleic acid molecule included in the co-formulation act together in the treatment of the disease or disorder; and a method of manufacturing the co-formulation.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 15, 2024, is named “177941.0067.xml” and is 52,829 bytes in size. The sequence listing contained in this .XML file is part of the specification and is hereby incorporated by reference herein in its entirety.

### FIELD OF THE DISCLOSURE

The present disclosure relates to co-formulation and co-delivery of a drug in combination with nucleic acid molecules. More particularly, the disclosure concerns the co-formulation of a small ionizable colloid forming molecule and a nucleic acid molecule, methods of co-delivering the co-formulation of the small ionizable colloid forming molecule and nucleic acid molecule, and to methods of using the co-formulation of the small ionizable colloid forming molecule and nucleic acid molecule in the treatment of diseases or disorders, and methods of manufacturing the co-formulations.

### BACKGROUND

Co-delivery of small molecule drugs and therapeutic RNA could allow synergistic treatment of diseases such as drug-resistant cancer¹. Specifically, RNA therapeutics can unlock targets currently undruggable by small molecule drugs². While synergistic compounds must act on the same cells, small molecule drugs and lipid-encapsulated nucleic acids distribute differently in the body³. Co-formulation can allow spatiotemporal co-localization of RNA and small molecule therapeutics. However, current delivery technologies are not optimized for co-formulation, with either sub-optimal RNA delivery for conventional liposomal or polymeric delivery systems used for small molecules or poor small molecule drug loading in RNA delivery vehicles (usually<10%)^{4,5}.

Many small molecule drugs self-assemble in aqueous solution to form drug-rich nanoparticles termed colloidal drug aggregates⁶. These nanoparticles can be stabilized with minimal excipients and modification of the surface with proteins or lipids enables cellular uptake via receptor-mediated endocytosis⁷. However, endosomal entrapment following uptake limits drug efficacy⁸. It has been previously demonstrated that the ionizable colloid-forming drug lapatinib, which is protonated during endosome acidification, escapes the endosome⁹. Furthermore, it has been shown that by chemically modifying colloid-forming fulvestrant with ionizable amine groups, the endosome is disrupted¹⁰.

### SUMMARY OF THE DISCLOSURE

In one embodiment, the present disclosure relates to a co-formulation comprising: (i) an ionizable colloid forming active substance; (ii) a nucleic acid molecule; and (iii) an amphiphilic molecule.

In one embodiment, the ionizable colloid forming active substance serves a dual role of (i) once ionized, complexing the nucleic acid molecule and forming a colloid nanoparticle entrapping the nucleic acid molecule therein and (ii) providing a therapeutic effect when the colloid nanoparticle is internalized.

In another embodiment of the co-formulation of the present disclosure, the ionizable colloid forming active substance is an ionizable analog of a non-ionizable molecule.

In another embodiment of the co-formulation of the present disclosure, the ionizable colloid forming active substance comprises an analog of an ionizable non-colloid forming active substance modified to form colloidal aggregates. In one aspect, the modification comprises addition of a piperidine, pyrrolidine or imidazole chemical group to the non-colloid forming active substance.

In another embodiment of the co-formulation of the present disclosure, the analog of the ionizable non-colloid forming active substance includes a hydrophobic group to form colloidal aggregates.

In another embodiment of the co-formulation of the present disclosure, the hydrophobic group is lauroyl, myristoyl, palmitoyl, stearoyl, oleoyl, linoleoyl, linolenoyl, or arachidonoyl acyl chains.

In another embodiment of the co-formulation of the present disclosure, the ionizable colloid forming active substance includes ionizable amine groups.

In another embodiment of the co-formulation of the present disclosure, the ionizable amine groups have experimentally determined pKa values ranging from 4 to 11.

In another embodiment of the co-formulation of the present disclosure, the nucleic acid molecule is a natural nucleic acid molecule or a xeno nucleic acid molecule.

In another embodiment of the co-formulation of the present disclosure, the nucleic acid molecule is a DNA molecule or RNA molecule.

In another embodiment of the co-formulation of the present disclosure, the DNA molecule or RNA molecule are functional DNA molecule or RNA molecule or decoy DNA molecule or RNA molecule.

In another embodiment of the co-formulation of the present disclosure, the functional DNA molecule or RNA molecule includes aptamers, miRNA, mRNA and siRNA.

In another embodiment of the co-formulation of the present disclosure, the ionizable colloid-forming active substance is derived from a non-ionizable, non-colloid forming molecule.

In another embodiment of the co-formulation of the present disclosure, the amphiphilic molecule is a polyethylene glycol-conjugated lipid (PEG-lipid), poly (carboxybetaine)-conjugated lipid, or combination thereof.

In another embodiment of the co-formulation of the present disclosure, the amphiphilic molecule is PEG-lipid.

In another embodiment of the co-formulation of the present disclosure, the amphiphilic molecule is cholesterol, a cholesterol analogue, a phospholipid, a phospholipid analogue, or a combination thereof.

In another embodiment of the co-formulation of the present disclosure, the amphiphilic molecule is cholesterol, a phospholipid, or a combination thereof.

In another embodiment of the co-formulation of the present disclosure, the amphiphilic molecule is a combination of cholesterol and phospholipid.

In another embodiment of the co-formulation of the present disclosure, the amphiphilic molecule further comprises PEG-lipid.

In another embodiment of the co-formulation of the present disclosure, the PEG-lipid is 1,2-dimyristoyl-rac-glycero-3-methoxypolyethylene glycol-2000.

In another embodiment of the co-formulation of the present disclosure, the phospholipid includes distearylphosphatidylcholine (DSPC), dilauroylphosphatidylcholine (DLPC), dipalmitoylphosphatidylcholine (DPPC), diarachidoylphosphatidylcholine (DAPC), dimyristoylphosphatidylcholine (DMPC), dioleoylphosphatidylcholine (DOPC), dioleoylphosphoethanolamine (DOPE), distearylphosphoethanolamine (DSPE), dilauroylphosphatidylserine (DLPS), dioleoylphosphatidylserine (DOPS) or any combination thereof.

In another embodiment of the co-formulation of the present disclosure, the co-formulation has a z-average hydrodynamic diameter of about 1000 nm or less, preferably 200 nm or less.

In another embodiment of the co-formulation of the present disclosure, the ionizable colloid forming active substance is an ionizable fulvestrant analog, and wherein the ionizable fulvestrant analog comprises fulvestrant having one or more ionizable amine groups.

In another embodiment of the co-formulation of the present disclosure, the active substance is one of netarsudil, amiodarone, emetine, lapatinib, levofloxacin, siramesine, an ionizable fulvestrant analog, bedaquiline, certinib, bazedoxifene, nilotinib, toremifene, elbasvir, isoconazole, bafetinib, clomiphene, pasireotide, sertaconazole, carvediol, afatinib, thioridazine, tamoxifen, or a combination thereof.

In another embodiment of the co-formulation of the present disclosure, the ionizable fulvestrant analog is combined with another active substance selected from the group consisting of fulvestrant, netarsudil, amiodarone, apilimod, lapatinib, levofloxacin, siramesine, bedaquiline, certinib, bazedoxifene, nilotinib, toremifene, elbasvir, isoconazole, bafetinib, clomiphene, pasireotide, sertaconazole, carvediol, afatinib, thioridazine and tamoxifen.

In another embodiment of the co-formulation of the present disclosure, the active substance is a sorafenib analog or an emetine analog.

In another embodiment of the co-formulation of the present disclosure, the co-formulation further comprises an ionizable lipid.

In another embodiment of the co-formulation of the present disclosure, the colloid forming ionizable active substance comprises emetine modified to form colloidal aggregates.

In another embodiment of the co-formulation of the present disclosure, the co-formulation is free of ionizable lipids.

In another embodiment, the present disclosure relates to a method of treating a disease or disorder comprising administering the co-formulation according to an embodiment of the present disclosure to a subject in need, wherein the ionizable colloid-forming active substance and the nucleic acid molecule included in the co-formulation act together in the treatment of the disease or disorder.

In one embodiment of the method of treating a disease or disorder of the present disclosure, the disease or disorder is cancer. In one aspect, the cancer is a drug-resistant cancer. In aspects of the disclosure, the cancer includes bladder cancer, cervical cancer, ovarian cancer, breast cancer, prostate cancer, testicular cancer, head and neck cancer, lung cancer, pancreatic cancer, stomach cancer, colorectal cancer, liver cancer, esophageal cancer, and brain cancer.

In another embodiment of the method of treating a disease or disorder of the present disclosure, the disorder or disease is leukemia. In aspects, the leukemia is drug resistant leukemia.

In another embodiment, the present disclosure relates to a use of the co-formulation according an embodiment of the present disclosure in the treatment of a disease or disorder, wherein the ionizable colloid-forming active substance and the nucleic acid molecule included in the co-formulation act together in the treatment of the disease or disorder.

In one embodiment of the use of the co-formulation of the present disclosure, the disease or disorder is cancer. In aspects, the cancer is a drug-resistant cancer.

In another embodiment of the use of the co-formulation, the cancer includes bladder cancer, cervical cancer, ovarian cancer, breast cancer, prostate cancer, testicular cancer, head and neck cancer, lung cancer, pancreatic cancer, stomach cancer, colorectal cancer, liver cancer, esophageal cancer, and brain cancer.

In another embodiment of the use of the co-formulation, the disorder or disease is leukemia. In aspects, the leukemia is drug resistant leukemia.

In another embodiment, the present disclosure relates to a method of manufacturing the co-formulation according to an embodiment of the present disclosure, wherein the method comprises: (a) mixing the ionizable colloid forming active substance with the nucleic acid molecule at a pH lower than the pKa value of the ionizable colloid forming active substance to form a particle having the nucleic acid molecule entrapped therein; (b) neutralizing the particle's surface charge in a buffer having neutral or close to neutral pH or a pH greater than the largest pKₐ value of the ionizable colloid forming active substance; and (c) adding an amphiphilic molecule to facilitate entrapment of the nucleic acid molecule within the formed colloid nanoparticle and/or to stabilize the formed colloid nanoparticle.

In another embodiment, the present disclosure provides for an ionizable analog of emetine comprising emetine modified to include a hydrophobic group.

In one embodiment of the ionizable analog of emetine of the present disclosure, the hydrophobic lipid group is an oleyl hydrocarbon tail.

In another embodiment of the ionizable analog of emetine of the present disclosure, the hydrophobic group is lauroyl, myristoyl, palmitoyl, stearoyl, oleoyl, linoleoyl, linolenoyl, or arachidonoyl acyl chains.

### BRIEF DESCRIPTION OF THE DRAWINGS

FIG. 1: Ionizable drug nanoparticles encapsulate siRNA or mRNA and facilitate delivery in cells and mice.

FIG. 2: Structure and pKₐ of ionizable fulvestrant analogs.

FIGS. 3A-3J: 3A Hydrodynamic diameter at day 0 and 2, 3B initial polydispersity index, 3C hydrodynamic diameter during storage at 4° C., 3D polydispersity index during storage at 4° C. (*multimodal following this time point), 3E siRNA encapsulation efficiency, 3F siRNA loading efficiency, 3G zeta potential, 3H zeta potential vs. pKₐ (p-value for non-zero slope by simple linear regression), and 3I cellular uptake in SKOV3 cells (n=4, ordinary two-way ANOVA with Sidak post-hoc test, ****p<0.0001, representative images in 3J) of nanoparticles containing ionizable fulvestrant analogs encapsulating siRNA employing DSPC as a stabilizing phospholipid.

FIGS. 4A-4G: 4A Hydrodynamic diameter at day 0 and 15, 4B polydispersity index, 4C siRNA encapsulation efficiency, 4D siRNA loading efficiency, 4E zeta potential, and 4F cellular uptake in SKOV3 cells (n=3, ordinary two-way ANOVA with Sidak post-hoc test, ****p<0.0001, representative images in 4G) of nanoparticles containing fulvestrant analog 7d encapsulating siRNA employing varying phospholipids.

FIGS. 5A-5J: 5A Hydrodynamic diameter at day 0 and 2, 5B initial polydispersity index, 5C hydrodynamic diameter during storage at 4° C., 5D polydispersity index during storage at 4° C., 5E siRNA encapsulation efficiency, 5F siRNA loading efficiency, 5G zeta potential, 5H zeta potential vs. pKₐ (p-value for non-zero slope by simple linear regression), and 5I cellular uptake in SKOV3 cells (n=4, ordinary two-way ANOVA with Sidak post-hoc test, *p<0.05, ***p<0.001, representative images in 5J) of nanoparticles containing ionizable fulvestrant analogs encapsulating siRNA employing DOPC as a stabilizing phospholipid.

FIG. 6: Cellular uptake of F_{X}NPs employing DOPC in SKOV3 cells after 3 h of treatment at 20 nM siRNA. Media was replaced with either serum free media, serum free media supplemented with ApoE (1.5 μg/mL) or complete media before treating with F_{X}NPs (n=4, ordinary two-way ANOVA with Sidak post-hoc test, *p<0.05, **p<0.01, ***p<0.001, ****p<0.0001).

FIG. 7: Sample fluorescence images of SKOV3 cells after 3 h of treatment with F_{X}NPs employing DOPC containing siFLuc at 20 nM. Media was replaced with either serum free media, serum free media supplemented with ApoE (1.5 μg/mL) or complete media before treating with F_{X}NPs. F_{X}NPs (DiD) are shown in the left column and nuclei (Hoechst) are shown in the right column.

FIGS. 8A-8B: Cryo-TEM images of F_{7f}NPs with DOPC at 8A 28,000× and 8B 57,000× magnification.

FIG. 9: Graph illustrating measured pKₐ values of different fulvestrant analog formulations (N=3, mean±SEM, two-way ANOVA with Tukey's post-hoc test comparing all formulations of each compound, *p<0.05, **p<0.01, ***p<0.001, ****p<0.0001).

FIGS. 10A-10D: Expression levels of 10A FLuc in SKOV3-FLuc cells or 10B nLuc in Vero-nLucP cells following treatment with F_{X}NPs containing ionizable fulvestrant analogs, DOPC, and targeting (siFluc for SKOV3-FLuc and sinLuc for Vero-nLucP) or non-targeting (sinLuc for SKOV3-FLuc and siFLuc for Vero-nLucP) siRNA. Cell metabolic activity for 10C SKOV3-FLuc or 10D Vero-nLucP cells after treatment with F_{X}NPs encapsulating targeting or non-targeting siRNA (n=3, ordinary two-way ANOVA with Sidak post-hoc test, *p<0.05, ***p<0.001, ****p<0.0001, ns=not significant).

FIGS. 11A-11E: 11A Hydrodynamic diameter, 11B polydispersity index, 11C siRNA encapsulation efficiency, 11D zeta potential, and 11E luciferase knockdown potency (half-maximal inhibitory concentration; IC₅₀) in SKOV3-FLuc cells for F_{7g}NPs employing either DOPC or DOPE, formulated in either 50 mM citrate, 25 mM citrate, 25 mM malate or 25 mM acetate, and encapsulating either siFLuc or non-targeting siRNA.

FIGS. 12A-12F: Cryogenic transmission electron microscopy images for F_{7g}NPs employing DOPC and formulated in 12A 50 mM citrate or 12B 25 mM acetate, or DOPE and formulated in 12C 50 mM citrate, 12D 25 mM citrate, 12E 25 mM malate, or 12F 25 mM acetate (scale bar=100 nm).

FIGS. 13A-13B: 13A Cellular uptake of F_{7g}NPs employing either DOPC or DOPE, formulated in either 50 mM citrate, 25 mM citrate, 25 mM malate or 25 mM acetate, and encapsulating either siFLuc or non-targeting siRNA in SKOV3 cells after 3 h of treatment at 20 nM siRNA. Media was replaced with either serum free media, serum free media supplemented with ApoE (1.5 μg/mL) or complete media before treating with F_{7g}NPs (n=6, ordinary two-way ANOVA with Sidak post-hoc test, ****p<0.0001). 13B Sample fluorescence images where nuclei (Hoechst) are shown in the left column, F_{X}NPs (DiD) are shown in the middle column and a merge of both are shown in the right column.

FIGS. 14A-14B: 14A Cellular uptake of F_{7g}NPs employing either DOPC or DOPE, formulated in either 50 mM citrate, 25 mM citrate, 25 mM malate or 25 mM acetate, and encapsulating either siFLuc or non-targeting siRNA in SKOV3 cells after 3 h of treatment at 20 nM siRNA. Media was replaced with either complete media or complete media supplemented with hydroxydynasore (20 μM) before treating with F_{7g}NPs (n=6, ordinary two-way ANOVA with Sidak post-hoc test, ****p<0.0001). 14B Sample fluorescence images where nuclei (Hoechst) are shown in the left column, F_{X}NPs (DiD) are shown in the middle column and a merge of both are shown in the right column.

FIGS. 15A-15F: 15A Hydrodynamic diameter, 15B polydispersity index, 15C siRNA encapsulation efficiency, 15D zeta potential, 15E luciferase knockdown potency (half-maximual inhibitory concentration; IC₅₀) in SKOV3-FLuc cells and 15F cellular metabolic activity at a 25 nM dose (n=3, mean±standard deviation, ordinary one-way ANOVA with Tukey post-hoc test, *p<0.05, ***p<0.001) for F_{7g}NPs formulated in 25 mM malate and employing DOPE with varying N/P ratios (3.0, 4.7 or 6.0).

FIGS. 16A-16B: 16A Cellular uptake of F_{7g}NPs employing either DOPC or DOPE, formulated in either 50 mM citrate, 25 mM citrate, 25 mM malate or 25 mM acetate, and encapsulating either siFLuc or non-targeting siRNA in T47D-R cells after 3 h of treatment at 20 nM siRNA. Media was replaced with either serum free media, serum free media supplemented with ApoE (1.5 μg/mL) or complete media before treating with F_{X}NPs (n=6, ordinary two-way ANOVA with Sidak post-hoc test, ****p<0.0001). 16B Sample fluorescence images where nuclei (Hoechst) are shown in the left column, F_{X}NPs (DiD) are shown in the middle column and a merge of both are shown in the right column.

FIGS. 17A-17B: 17A Cellular uptake of F_{7g}NPs employing either DOPC or DOPE, formulated in either 50 mM citrate, 25 mM citrate, 25 mM malate or 25 mM acetate, and encapsulating either siFLuc or non-targeting siRNA in T47D-R cells after 3 h of treatment at 20 nM siRNA. Media was replaced with either complete media or complete media supplemented with hydroxydynasore (20 μM) before treating with F_{X}NPs (n=6, ordinary two-way ANOVA with Sidak post-hoc test, ****p<0.0001). 17B Sample fluorescence images where nuclei (Hoechst) are shown in the left column, F_{X}NPs (DiD) are shown in the middle column and a merge of both are shown in the right column.

FIGS. 18A-18B: Expression of 18A CCNE1 and 18B GREB1 mRNA in T47D-R cells measured by qPCR following treatment with PBS, F_{7g}NPs encapsulating siRNA targeting CCNE1 or F_{7g}NPs encapsulating non-targeting siRNA (n=3 biological replicates, mean±standard deviation, ordinary one-way ANOVA with Tukey post-hoc test, *p<0.05, ***p<0.001, ****p<0.0001).

FIGS. 19A-19G: Fulvestrant analogs 7f and 7g can be used to deliver functional mRNA. 19A Hydrodynamic diameter, 19B polydispersity and 19C encapsulation efficiency for F_{7f}NPs and F_{7g}NPs encapsulating mFLuc. 19D Cell metabolic activity and 19E normalized firefly luciferase expression for HepG2 cells treated with varying amounts of F_{7f}NPs or F_{7g}NPs encapsulating mFLuc. Liver bioluminescence following i.v. injection of F_{7f}NPs or F_{7g}NPs in mice with 19F images and 19G quantification (n=2-4, ordinary two-way ANOVA with Sidak post-hoc test, *p<0.05, **p<0.01, ***p<0.001, ****p<0.0001).

FIGS. 20A-20C: Some existing ionizable drugs were identified that can form nanoparticles that encapsulate siRNA. 20A Hydrodynamic diameter and 20B encapsulation efficiency for nanoparticle formulations of various ionizable drugs encapsulating siRNA. 20C Chemical structures of ionizable drugs tested in siRNA nanoparticle formulations.

FIGS. 21A-21E: Existing drugs can be chemically modified to increase the propensity for siRNA encapsulation. 21A Hydrodynamic diameter and 21B encapsulation efficiency for nanoparticle formulations of sorafenib or an ionizable analog of sorafenib encapsulating siRNA. 21C Hydrodynamic diameter and 21D encapsulation efficiency for nanoparticle formulations of emetine or an analog of emetine with increased hydrophobicity encapsulating siRNA. 21E Chemical structures of parent drugs and analogs.

FIGS. 22A-22B: Cholesterol is essential for RNA encapsulating formulations. 22A Hydrodynamic diameter and 22B encapsulation efficiency for formulations with siramesine encapsulating siRNA with a lipid composition of 50:38.5:10:1 siramesine:cholesterol:DSPC:DMG-PEG-2000 (standard formulation) or 88.5:10:1 siramesine:DSPC:DMG-PEG-2000 (no cholesterol).

FIGS. 23A-23B: 23A Hydrodynamic diameter and 23B encapsulation efficiency for F_{7f}NPs encapsulating siRNA with varying stabilizing lipid compositions.

FIG. 24: Nanoparticle uptake in cells is influenced by the selection of phospholipid. Cellular uptake of DiD-labeled siramesine nanoparticles encapsulating RNA derived from Torula utilis yeast with varying phospholipid identity. SKOV3 cells were incubated with nanoparticles containing 20 nM RNA for 3 h (*P<0.05, ***P<0.001, ordinary one-way ANOVA with Dunnett's post-hoc test comparing all groups to DSPC).

FIGS. 25A-25D: Ionizable drugs can be used to form nanoparticles encapsulating multiple types of RNA. 25A Hydrodynamic diameter and 25B encapsulation efficiency for siramesine nanoparticle formulations encapsulating varying RNA cargos. 25C Hydrodynamic diameter and 25D encapsulation efficiency for fulvestrant analog 7f nanoparticle formulations encapsulating varying RNA cargos. T.U.=Torula Utilis (siRNA: ˜13.3 kDa, T.U. RNA: ˜5-8 kDa, rRNA: 551 & 990 kDa, mRNA: ˜618 kDa).

FIGS. 26A-26F: 26A Schematic for small scale screening method used to measure siRNA complexation by ionizable drugs. Comparison of complexation measured by small scale method and encapsulation efficiency in lipid nanoparticle (LNP) formulations for 26B various drugs and 26C fulvestrant analogs. 26D Normalized fluorescence data for screening of 215 drugs in small scale assay, with normalization to wells with no ionizable drug (a value of 1 represents no siRNA complexation and 0 represents complete siRNA complexation). Drugs were classified as siRNA complexing (relative fluorescence<0.5) or non-complexing (relative fluorescence>0.5) and combined with data for other ionizable drugs and fulvestrant analogs for a total set of 229 to fit using a random forest model. Confusion matrices for this model are shown for the 26E training set (80%) and 26F testing set (20%).

FIGS. 27A-27H: 27A Hydrodynamic diameter, 27B polydispersity index, and 27C siRNA encapsulation efficiency for DSPC-containing formulations of 16 hits from the drug screening assay. 27D Hydrodynamic diameter, 27E polydispersity index, and 27F siRNA encapsulation efficiency for formulations with DOPE of 5 best performing drugs. 27G FLuc expression and 27H cell metabolic activity in SKOV3-FLuc cells after treatment with DOPE-containing formulations encapsulating siFLuc or non-targeting siRNA (siNT).

FIGS. 28A-28C: 28A Chemical structure of netarsudil. 28B Cellular uptake of netarsudil nanoparticles encapsulating siRNA in SKOV3-FLuc cells. 28C Expression levels of FLuc in SKOV3-FLuc cells following treatment with netarsudil nanoparticles encapsulating targeting (siFLuc) or non-targeting (si5) siRNA.

FIGS. 29A-29L: 29A Hydrodynamic diameter, 29B polydispersity index, 29C siRNA encapsulation efficiency and 29D luciferase knockdown potency in SKOV3-FLuc cells for netarsudil nanoparticles neutralized in TBS with 50 mol % netarsudil, or 40 mol % netarsudil with 10 mol % of MC3, ALC-0315 or SM-102. 29E Hydrodynamic diameter, 29F polydispersity index, 29G siRNA encapsulation efficiency and 29H luciferase knockdown potency in SKOV3-FLuc cells for netarsudil nanoparticles neutralized in TBS with varying ratios of netarsudil to SM-102. 29I Hydrodynamic diameter, 29J polydisperity index, 29K siRNA encapsulation efficiency and 29L luciferase knockdown potency in SKOV3-FLuc cells for netarsudil nanoparticles neutralized in TBS with 10 mol % SM-102 and varying ratios of netarsudil to cholesterol.

### DETAILED DESCRIPTIONS OF THE DISCLOSURE

### Definitions

In this specification and in the claims that follow, reference will be made to a number of terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

Throughout this specification and the claims, the terms “comprise,” “comprises,” and “comprising” are used in a non-exclusive sense, except where the context requires otherwise. Likewise, the terms “include”, “has” and their grammatical variants are intended to be non-limiting, such that recitation of items in a list is not to the exclusion of other like items that can be substituted or added to the listed items. “Consisting essentially of” when used to define systems, compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers. “Consisting of” shall mean excluding more than trace elements of other ingredients and substantial method steps for using the multifunctional nanoparticles of the present disclosure. Embodiments defined by each of these transition terms are within the scope of this invention.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range.

The term “about,” particularly in reference to a given quantity, is meant to encompass deviations of plus or minus five percent.

“Active substance” or “drug” refers to a small molecule having a therapeutic effect.

“Co-delivery” refers to the simultaneous delivery of two or more drugs or agents. Delivery of a co-formulation falls within the definition of “co-delivery.”

“Co-formulation” refers to a product or composition in which two or more separate drug components are combined in a single dosage form.

“Effective amount” refers to an amount of the composition that is capable of producing a medically desirable result in a treated subject. The methods of the present disclosure may be performed alone or in combination with other drugs or therapies.

In this document, “ionizable active substance” or “ionizable drug” include ionizable active substances or drugs and also include ionizable analogs of non-ionizable active substances or drugs. The ionizable analog of the non-ionizable active substance or drug keeps the therapeutic effect of the original non-ionizable active substance or drug being modified or is a prodrug that regains its therapeutic effect upon cleavage. Ionizable active drug is ionizable to have a positive charge and become cationic as the pH is decreased.

In this document “colloid forming” active substances or drugs include active substances or drugs that naturally form colloids in aqueous solution, and also to active substances or drugs that do not readily form colloidal drug aggregates but are chemically modified to allow colloid formation. The modified active substance or drug keeps the therapeutic effect of the original non-colloid forming active substance or drug being modified or is a prodrug that regains its therapeutic effect upon cleavage.

In this document, “loading”, or “load” refers to a measure of how much therapeutic is incorporated into a nanoparticle on a mass basis.

“Pharmaceutically acceptable” refers to molecular entities and other ingredients of such compositions that are physiologically tolerable and do not typically produce unwanted reactions when administered to a subject.

The present invention relates to the co-formulation for co-delivery of small molecule drugs and nucleic acid-based drugs or therapeutics, such as DNA, siRNA-, miRNA-, mRNA-based drugs or therapeutics. In this document, the terms “drug(s)”, “therapeutic(s)”, “active agent(s)”, “active substance(s)” are used interchangeably.

A “nucleic acid drug” refers to a nucleic acid molecule having a therapeutic effect.

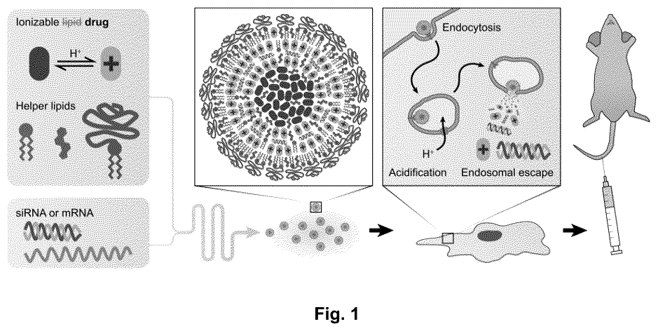

In the present disclosure, the self-assembling and endosome disruption behavior of specific small molecule drugs is being harnessed for intracellular delivery of a nucleic acid molecule. To achieve endosomal escape, the acidic environment of the endo-lysosomal pathway for drug protonation is being leveraged by co-formulating ionizable drugs and nucleic acid molecules, including nucleic acid drugs. It has been found that colloid forming ionizable molecules having therapeutic activity facilitate endosomal escape of co-delivered nucleic acid. The colloid forming ionizable drug itself facilitates nucleic acid encapsulation. The present disclosure captures any molecule that is ionizable, colloid forming and has a therapeutic activity. This novel combination strategy allows co-delivery of a small molecule drug and a nucleic acid molecule in the same nanoparticle with spatiotemporal control (see FIG. 1).

In one embodiment of the present disclosure, the nucleic acid is a natural nucleic acid or a xeno nucleic acid. In another embodiment of the present disclosure, the nucleic acid is DNA or RNA. In another embodiment of the present disclosure, the DNA or RNA are functional DNA or RNA or decoy DNA or RNA. In another embodiment of the present disclosure, the functional DNA or RNA include aptamers, plasmids, miRNA, mRNA and siRNA.

In one embodiment, the nucleic acid molecule has a therapeutic activity (also referred to as nucleic acid drug). In another embodiment, the nucleic acid molecule has a use in research.

In embodiments of the present disclosure, the ionizable lipid component in existing LNP (“lipid nanoparticle”) platforms is replaced partially or completely with a colloid forming ionizable drug for nucleic acid delivery. This strategy can be applied to a range of nucleic acid cargos. Most drugs in the Merck Index are weakly basic¹¹ and many spontaneously form nanoparticles in aqueous media⁶. Thus, the approach of the present disclosure is broadly applicable to a range of small molecule drug therapeutics.

In one embodiment, the present disclosure relates to a co-formulation comprising: (i) an ionizable colloid forming active substance; (ii) a nucleic acid molecule; and (iii) an amphiphilic molecule.

The ionizable, colloid forming active substance of the co-formulation once ionized complexes the nucleic acid molecule and forms a colloid nanoparticle entrapping the nucleic acid therein, and the amphiphilic molecule facilitates the encapsulation of the nucleic acid molecule within the formed colloid nanoparticle and/or stabilizes the formed colloid nanoparticle through maintaining integrity of the particle structure and/or sterically stabilizing the formed colloid nanoparticle thereby preventing nanoparticle aggregation. In one embodiment, the nucleic acid molecule is a nucleic acid drug. In another embodiment, the nucleic acid molecule is for research purposes.

The ionizable colloid forming active substance serves a dual role of (i) once ionized in aqueous solution, complexing the nucleic acid and forming a colloid nanoparticle entrapping the nucleic acid therein and (ii) providing a therapeutic effect when the colloid nanoparticle is internalized in cells.

The present disclosure extends to colloid forming ionizable drugs having a suitable degree of nucleic acid molecule encapsulation efficiency, for example, of about 5% or more, or about 10% or more, or about 15% or more, or about 20% or more, or about 30% or more, or about 40% or more, or about 50% or more, or about 60% or more, or about 70% or more, or about 80% or more, or about 90% or more.

The present disclosure extends to colloid forming ionizable drugs having a suitable degree of nucleic acid molecule loading efficiency, for example, of about 1% or more, or about 2% or more, or about 3% or more, or about 4% or more, or 5% or more, or about 10% or more, or about 15% or more.

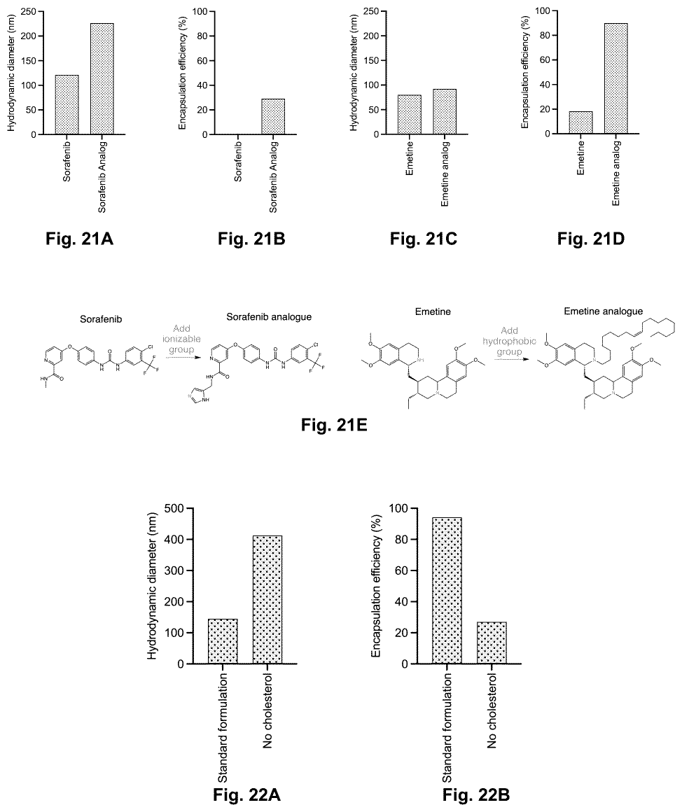

In one embodiment, the present disclosure extends to ionizable drugs and to ionizable analogs of non-ionizable drugs. Chemical modification of non-ionizable drugs to ionizable drugs resulted in encapsulation and loading of siRNA molecules, as shown in FIG. 21B, while chemical modification of ionizable drugs with hydrophobic moieties also resulted in siRNA encapsulation (FIG. 21D).

In another embodiment, the present disclosure relates to colloid forming ionizable drugs and to ionizable drugs that do not readily form colloidal aggregates, but which are modified so that they can form colloidal aggregates. For example, emetine is ionizable but does not readily form colloidal drug aggregates. Modification of emetine with a hydrophobic tail to increase hydrophobicity allows colloid formation and improves siRNA encapsulation (FIGS. 21C, 21D).

As such, in another embodiment, the present disclosure extends to ionizable colloid forming analogs of non-ionizable, non-colloid forming drugs.

In another embodiment, the present disclosure also provides methods of using the co-formulations of the present disclosure in the treatment of conditions, diseases or disorders that are the targets of the colloid forming ionizable drug and the nucleic acid therapeutic included in the co-formulation. The co-delivery of the colloid forming ionizable drug and nucleic acid molecules, including nucleic acid drugs (such as siRNA drugs) allow synergistic treatment of diseases such as cancer, in particular drug-resistant cancer. Cancers (including drug-resistant cancers) that can be treated with the co-formulation of the present disclosure include bladder cancer, cervical cancer, ovarian cancer, breast cancer, prostate cancer, testicular cancer, head and neck cancer, lung cancer, pancreatic cancer, stomach cancer, colorectal cancer, liver cancer, esophageal cancer, and brain cancer. Also, leukemia and drug resistant leukemia can be treated with the co-formulation of the present disclosure.

In another embodiment, the present disclosure relates to a method of manufacturing the co-formulations of the present disclosure. In one embodiment, the method comprises: (a) mixing an ionizable colloid-forming active substance with a nucleic acid molecule at a pH lower than the pKa value of the ionizable colloid-forming active substance to form a particle having the nucleic acid drug entrapped therein; (b) neutralizing the particle's surface charge in a buffer having neutral or close to neutral pH; and (c) adding an amphiphilic molecule to facilitate entrapment of the nucleic acid molecule within the formed colloid nanoparticle and/or to stabilize the formed colloid nanoparticle.

In order to aid in the understanding and preparation of the present disclosure, the following illustrative, non-limiting examples are provided.

### EXAMPLES

We start by demonstrating the utility of ionizable fulvestrant analogs for siRNA and mRNA delivery. Then, other ionizable drugs, including those that natively include ionizable groups and those that are modified to include ionizable groups, are investigated. This work demonstrates RNA delivery by ionizable drugs in a co-formulation. This is the foundation of our invention.

### Example 1

### Preparation and Characterization of Ionizable Drug Nanoparticles Encapsulating RNA

Nanoparticles were formulated using a NanoAssemblr Benchtop microfluidic mixing instrument (Precision NanoSystems) with a composition of ionizable drug to cholesterol to phospholipid to DMG-PEG-2000 to DiD at a molar ratio of 50:38.5:10:1.5:0.1, unless otherwise specified. siFLuc (sense (SEQ ID NO: 1): 5′-UCGAAGUACUCAGCGUAAGdTdT-3′ antisense (SEQ ID NO: 2): 5′-CUUACGCUGAGUACUUCGAdTdT-3′ where “d” denotes a DNA base), siFLuc-2 (sense (SEQ ID NO: 3): 5′-mGmGUmUCmCUGGAAmC-AmAUmUGmCUUUUAmCdA-3′ antisense (SEQ ID NO: 4): 5′-UGMUAAAAGmCAmAUmU-GUUCCAGGAmACmCmAmG-3′ where “d” denotes a DNA base and “m” indicates a 2′-O-methyl RNA base), sinLuc (sense (SEQ ID NO: 5): 5′-GGAUUGUCCUGAGCGGUGAdTdT-3′ antisense SEQ ID NO: 6): 5′-UCACCGCUCAGGACAAUCCdTdT-3′ where “d” denotes a DNA base), siCCNE1 (sense (SEQ ID NO: 7): 5′-mGmCUmUCmGGCCUUm-GUmAUmCAmUUUCUCmGT-3′ antisense (SEQ ID NO: 8): 5′-ACmGAGAAAmUGmAUmA-CAAGGCCGAmAGmCmUmG-3′ where “d” denotes a DNA base and “m” indicates a 2′-O-methyl RNA base), siNT (sense (SEQ ID NO: 9): 5′-mAmUAmCCmUUCCCAmG-GmUAmACmAAACCAmAdC-3′ antisense (SEQ ID NO: 10): 5′-GUmUGGUUUmGUmUAmC-CUGGGAAGGmUAmUmAmA-3′ where “d” denotes a DNA base and “m” indicates a 2′-O-methyl RNA base), mFLuc (EZ Cap Firefly Luciferase mRNA (5-moUTP), ApexBio), rRNA or RNA from Torula utilis yeast (T.U. RNA) was diluted in citrate buffer (50 or 25 mM, pH 4), malate buffer (25 mM, pH 4.0) or acetate buffer (25 mM, pH 4.0). A lipid/drug mix concentration of 5.5 mM was used with a total flow rate of 9 mL/min at a 3:1 ratio of aqueous to organic phase. N/P ratios between 3 and 6 between ionizable drug amines (N) and siRNA phosphates (P) were chosen, which is within the range of N/P values typically employed for LNPs used to deliver siRNA or mRNA of 1-12¹². After formulation, buffer exchange into PBS or Tris-buffered saline (TBS; pH 8.5) was performed using Amicon Ultra-4 centrifugal filters (molecular weight cut-off 10 kDa). Hydrodynamic diameter, polydispersity index (PDI), and scattering intensity were measured by dynamic light scattering (DLS) using a DynaPro Plate Reader II (Wyatt Technologies). The instrument was configured with a 60 mW 830 nm laser and a detector angle of 158°. A 25 μL sample of each formulation was pipetted into each well of a 384-well plate and measured with 20 acquisitions per sample at 25° C. Encapsulation efficiency of siRNA was determined using a modified Quant-iT RiboGreen assay as previously described.¹³ Briefly, nanoparticles were diluted in Tris-EDTA (TE) buffer with (total siRNA) or without (unencapsulated siRNA) 1% Triton X-100. Quant-iT RiboGreen reagent was added, and fluorescence intensity was quantified using a Tecan Infinite Pro 200 plate reader. Encapsulation efficiency was calculated using eq. 1

$$EE = [ 1 - \frac{Unencapsulated siRNA}{Total siRNA} ] \times 100 \% \qquad 1$$

Zeta potential was measured by diluting F_{X}NPs in phosphate to an ion concentration of 1 mM and using a Malvern Zetasizer Nano ZS instrument.

### pKₐ Determination

Formulation pKₐ values were measured by a previously described fluorescence assay.^{14,15} Solutions with 1.2 mM 2-(p-toluidino) naphthalene-6-sulfonic acid (TNS) and 2.0 mM F_{X}NPs (in terms of ionizable drug) in DMSO were prepared. 2 μL of this solution was mixed with 200 μL of PBS (pH adjusted between 4.0-10.0). TNS fluorescence (λₑₓ=322 nm, λₑₘ=431 nm) was measured using a plate reader. pKₐ values were determined by non-linear regression with fluorescence versus pH data with eq. 2

$$Fluorescence = Background + \frac{Maximum - Background}{1 + {10}^{pH - {pK}_{a}}} \qquad 2$$

### Cryo-TEM

F_{X}NPs were concentrated to a final concentration of 20-25 mg/mL of total lipid. 4 μL of each LNP suspension was added to glow-discharged (PELCO easiGlow, Ted Pella, Inc.) Lacey Formvar/carbon copper grids (Ted Pella, Inc., Redding, CA) and vitrified in liquid ethane using a FEI Mark IV Vitrobot (Thermo Fisher Scientific). Grids were stored in liquid nitrogen until imaging. Grids were transferred into a transmission electron microscope single-tilt cryo-holder (Gatan, Inc.). A Talos L120C (Thermo Fisher Scientific) with a high tension of 120 kV and a 4×4 k BM-Ceta CMOS camera was used to image samples. Samples were imaged at 27,000×, 57,000× or 120,000× magnification, yielding a pixel size of 510, 249, and 121 pm, respectively. Sample preparation and imaging were performed at the University of Toronto Microscopy Imaging Laboratory (Toronto, ON). Noise removal was performed using the “Remove Outliers” tool with Fiji software.¹⁶

### Cell Culture

SKOV3 cells were obtained from ATCC. SKOV3-luc cells were obtained from Cell Biolabs. Vero-E6-nLucP cells were a generous gift from Roman Melnyk. T47D cells were a gift from B. Neel (University Health Network). Palbociclib-resistant T47D cells (T47D-R) were developed by dose escalation of palbociclib up to 500-1000 nM over 6-12 months and was confirmed by a palbociclib IC50 greater than 1 M. Parental T47D cells (T47D-P) were cultured over the same time frame without palbociclib treatment. Cells were maintained in a humified incubator at 37° C. with 5% atmospheric CO₂. RPMI 1640 (SKOV3, T47D) or EMEM (Vero-E6) were used as a base media. Cells were grown in 75 cm² tissue culture flasks with 10 mL of media supplemented with 10% FBS, 10 UI/mL penicillin and 10 μg/mL streptomycin. The cells were passaged once per week following detachment with trypsin-EDTA, replacement of the supernatant with fresh media, and subculture into a new flask with fresh media. Subculture ratios between 1:4 and 1:50 were used.

### Cellular Uptake

2.5-5.0×10³ SKOV3 cells or 1.0×10⁴ T47D cells in 25 μL were plated in each well of a 384-well plate (Greiner Bio-One 781097) and allowed to adhere overnight. For endocytosis blocking studies, cells were washed once with Hank's Balanced Salt Solution (HBSS), and then fresh media containing an endocytosis blocker was added and plates were incubated at 37° C. for 30 min prior to dosing. For ApoE supplementation studies and endocytosis-blocking studies, serum free media, serum free media supplemented with ApoE (1.5 μg/mL), complete media, or complete media with hydroxydynasore (20 μM) was added for 30 min prior to dosing). Nanoparticle suspensions were diluted in PBS and 5 μL of each tested formulation was dosed in triplicate, followed by 20 μL of complete cell growth media (or media containing an endocytosis blocker or ApoE, according to the given treatment condition) to promote mixing. The final siRNA concentration in each well was between 2.5 and 40 nM. The plate was incubated at 37° C. for 3 h after which the media was removed, and the cells were washed with HBSS, fixed for 15 min with 4% (m/v) PFA in PBS, and stained for 15 min with 5 μg/mL Hoechst in PBS before imaging by wide-field fluorescence microscopy imaging as described below.

### Image Acquisition

Fluorescence images were acquired using a Zeiss Apotome Live Cell System (Axio Observer Z.1 inverted fluorescence microscope) with a long working distance 40× Plan Neofluor objective (NA=0.6, Carl Zeiss Canada), and X-Cite 120 LED fluorescence lamp (Lumen Dynamics), and an

Axiocam 506 mono camera (Carl Zeiss Canada). Focal plane selection was automated based on the nuclei channel (Hoechst). Four tiles per well were collected and stitched into a single image. An excitation band of 359-371 nm and an emission band of >397 nm were used for Hoechst 33342. An excitation band of 625-655 nm and an emission band of 665-715 nm were used for DiD. Illumination was carried out at 100% laser power with exposure times of 200 ms for Hoechst 33342 and 500 ms for DiD. Illumination and detector settings were held constant across different wells and plates.

### Image Processing

Image processing was performed with MATLAB based on an algorithm originally developed by Kameron Kilchrist.¹⁷ The modified code can be found on GitHub (github.com/kaislaughter/mChG8_image_processing). The main purpose of this code is to identify and count cell nuclei and endocytosed colloidal drug aggregates. First, images were treated with a top hat transform and threshold to remove background fluorescence. Next, the images were binarized, and a watershed algorithm was applied to split up partially overlapping features, such as two side-by-side nuclei. Finally, the number of features were counted and tabulated. The results of three images, each originating from a separate well, were averaged to yield the value for each biological replicate.

### Model Gene Knockdown

For single-dose studies, 5×10³ SKOV3-FLuc or 2×10³ Vero-nLucP cells in 100 μL were plated in each well of a white-wall 96-well plate (Costar 3610) and allowed to adhere overnight. F_{X}NP suspensions were diluted in PBS and 10 μL of each tested formulation was dosed in triplicate, followed by 100 μL of complete cell growth media to promote mixing. The final encapsulated siRNA concentration in each well was 50.0 nM. For dose-response studies, 1.25×10³ SKOV3-FLuc cells in 25 μL were plated in each well of a white-wall 384-well plate (Greiner Bio-one 781073) and allowed to adhere overnight. Nanoparticle suspensions were diluted in PBS and 5 μL of each tested formulation was dosed in triplicate, followed by 20 μL of complete cell growth media to promote mixing. The final encapsulated siRNA concentration in each well is specified in each figure. For all studies, the plate was incubated for 24 h followed by removal of the media. For SKOV3-FLuc cells, fresh media was added, and the plate was incubated for an additional 24 h before removal of the media. Total cellular metabolic activity was quantified by addition of PrestoBlue™ according to the manufacturer's instructions, after which the reagent was subsequently removed, and expression of either firefly luciferase (SKOV3-FLuc) or nano luciferase (Vero-nLucP) was determined by Steady-Glo® Luciferase Assay System or Nano-Glo® Luciferase Assay System, respectively. Luminescence was measured using a Tecan Infinite Pro 200 plate reader. Cell metabolic activity and reporter gene expression are represented relative to cells treated with an equivalent volume of PBS. Reporter gene expression is normalized to cell metabolic activity. For dose-response studies, IC₅₀ values were determined by non-linear regression analysis using eq. 3

$$Normalized luminescence = \frac{100 \%}{1 + {( \frac{{IC}_{50}}{[ siRNA ]} )}^{n}} \qquad 3$$

where n is the Hill slope (shared between groups).

### qPCR

1.0×10⁵ parental or palbociclib-resistant T47D cells in 500 μL were plated in each well of a 24-well plate and allowed to adhere overnight. F_{X}NP suspensions were diluted in PBS and 50 μL of each tested formulation was dosed in triplicate at a concentration of 20 nM siRNA. For siRNA validation studies, siRNAs were diluted in Opti-MEM and mixed with Lipofectamine-RNAiMAX Transfection Reagent (Invitrogen, Thermo Fisher Scientific, Waltham, MA) according to manufacturer instructions and 50 μL was added to in triplicate to wells. 500 μL of media was subsequently added to promote mixing. After 24 h, treatments were removed, wells were washed with media and 500 μL of fresh media was added. After an additional 24 h of incubation, cells were detached with trypsin-EDTA, pelleted by centrifugation (300×g, 5 min), washed with 1 mL PBS, and pelleted again (300×g, 5 min). PBS was aspirated and cell pellets were flash frozen in liquid nitrogen for storage until processing for qPCR analysis. Total RNA was extracted from cell pellets using a NucleoSpin RNA II Kit (Macherey-Nagel) according to manufacturer instructions. Reverse transcription was performed with a SuperScript VILO cDNA Synthesis Kit (Thermo Fisher Scientific, Waltham, MA), according to the manufacturer's protocol. qPCR was performed using a PowerUp SYBR Master Mix (Thermo Fisher Scientific, Waltham, MA) with fast cycling mode thermal cycling conditions as per manufacturer instructions using a QuantStudio™ 6 Flex System, and primer sequences summarized in Table 1. Primers were purchased from Integrated DNA Technologies (Coralville, IA). Fold change was calculated using the 2^{-ΔΔC}^{_{T}} method.

TABLE 1
Primers used in qPCR.

| Gene | SEQ ID NO | Primer | Sequence |
|---|---|---|---|
| CCNE1¹⁸ | 11 | Forward | 5′-TGTGTCCTGGATGTT |
| | | | GACTGCC-3′ |
| | 12 | Reverse | 5′-CTCTATGTCGCACCA |
| | | | CTGATACC-3′ |
| GREB1¹⁹ | 13 | Forward | 5′-CAAAGAATAACCTGT |
| | | | TGGCCCTGC-3′ |
| | 14 | Reverse | 5′-GACATGCCTGCGCTC |
| | | | TCATACTTA-3′ |
| ACTB²⁰ | 15 | Forward | 5′-CCAACCGCGAGAAGA |
| | | | TGACCCAGATCATGT-3′ |
| | 16 | Reverse | 5′-GTGAGGATCTTCATG |
| | | | AGGTAGTCAGTCAGG-3′ |

### Transfection With mRNA Formulations

5×10³ SKOV3 cells in 100 μL were plated in each well of a white-wall 96-well plate (Costar 3610) and allowed to adhere overnight. F_{X}NP suspensions were diluted in PBS and 10 μL of each 5 tested formulation was dosed in triplicate, followed by 100 μL of complete cell growth media to promote mixing. The final encapsulated mRNA amount in each well ranged from 12.5 to 100 ng. The plate was incubated for 24 h followed by removal of the media. Total cellular metabolic activity was quantified by addition of PrestoBlue™ according to the manufacturer's instructions, reagent was removed, and expression of firefly luciferase was determined by Steady-Glo® Luciferase Assay System. Luminescence was measured using a Tecan Infinite Pro 200 plate 10 reader. Cell metabolic activity and reporter gene expression are represented relative to cells treated with an equivalent volume of PBS. Reporter gene expression is normalized to cell metabolic activity.

### In Vivo Studies

Animal study protocols were approved by the Animal Care Committee at the University of Toronto and experiments were performed in accordance with the Guide to Care and Use of Experimental Animals (Canadian Council on Animal Care). F_{X}NPs with encapsulated mFLuc and DIR (0.5 mol %) were administered (1 mg/kg in terms of mRNA) via tail vein injection to BALB/c mice. After 4 h and 24 h, mice were anesthetized using isoflurane and injected intraperitoneally with 150 mg/kg VivoGlo™ luciferin (Promega, Madison, WI). Whole-body luminescence was acquired by a PerkinElmer In-Vivo Imaging System Spectrum instrument after 10-20 minutes.

### Small Scale siRNA Complexation Screening

Drugs were purchased from MedChemExpress pre-dissolved in DMSO at 10 mM in 96 well plates. 1 μL of each drug solution was added to 2 wells in a 96 well plate, followed by addition of 99 μL of siRNA in acetate (25 mM, pH 4.0). The siRNA concentration in acetate was selected for a final N/P ratio of 6. Additional wells were prepared with only DMSO and acetate (blank) or DMSO and siRNA in acetate (control for no complexation). Fluorescence was measured using a Tecan Infinite Pro 200 plate reader to check for possible drug fluorescence that would interfere with RNA detection by RiboGreen dye. 100 μL of diluted RiboGreen dye in acetate buffer was added to each well. RiboGreen fluorescence was measured to assess siRNA complexation by drugs. Fluorescence was normalized to wells with siRNA but no drug. Finally, complexes were disrupted with 0.5% Triton, or 0.5% Triton and 0.5 mg/mL heparin, and fluorescence was measured again to confirm any decrease in fluorescence could be attributed to siRNA complexation.

### Computational Modelling

Chemical structures in SMILES representation and selected chemical properties of drugs were extracted from DrugBank version 5.1. Drugs were selected for the small scale screening described above using the following criteria: (1) predicted log P>3, (2) predicted pKa (strongest basic) between 4 and 11, (3) physiological charge≥0 and (4) predicted to form colloidal drug aggregates. Colloidal drug aggregate formation prediction was performed by cross referencing with a list of drugs from DrugBank predicted to form colloidal drug aggregates reported by Reker et al.²¹ Following the drug screening assay, data was pre-processed to remove anomalous results including: (1) drugs that were fluorescent without addition of RiboGreen, (2) drugs that reduced fluorescence that couldn't be regained upon adding complex disruption agents and (3) drugs that had initially high fluorescence with siRNA but significantly reduced fluorescence upon adding complex disruption agents. Normalized fluorescence values were then used to classify drugs as those that could complex siRNA (“True”, normalized fluorescence<0.5) or those that could not (“False”, normalized fluorescence>0.5) to facilitate training of a binary classification model. 5666 molecular descriptors from the alvaDesc descriptor set were generated for each drug using the Online chemical modelling environment (OCHEM, ochem.eu)²². This molecular descriptor set was reduced by removing descriptors that shared the same value for all compounds or were strongly correlated to each other (Pearson r>0.95). This reduced descriptor set and siRNA complexation data for each assayed drug was used as input for a random forest classification machine learning model with 100 trees and a maximum of 10 features per classification tree. Compounds were randomly divided into stratified training (80%) and testing (20%) sets, where the training set was used to fit the model and the testing set was used to estimate its performance on new compounds. Model performance was assessed by calculating balanced accuracy (eq. 4)

$$Balanced accuracy = \frac{1}{2} ( \frac{True positives}{True positives + False negatives} + \frac{True negatives}{True negatives + False positives} ) \qquad 4$$

### Results

### siRNA Encapsulation and Delivery by Ionizable Fulvestrant Analogs

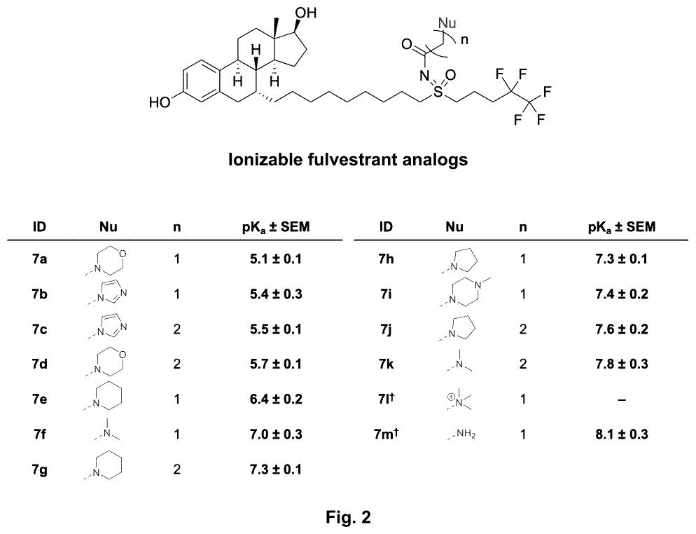

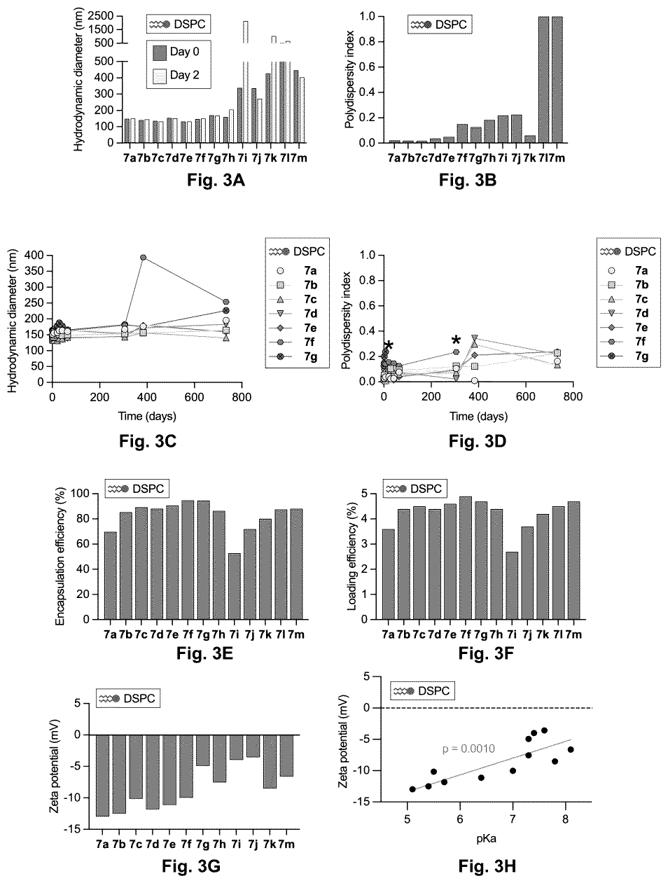

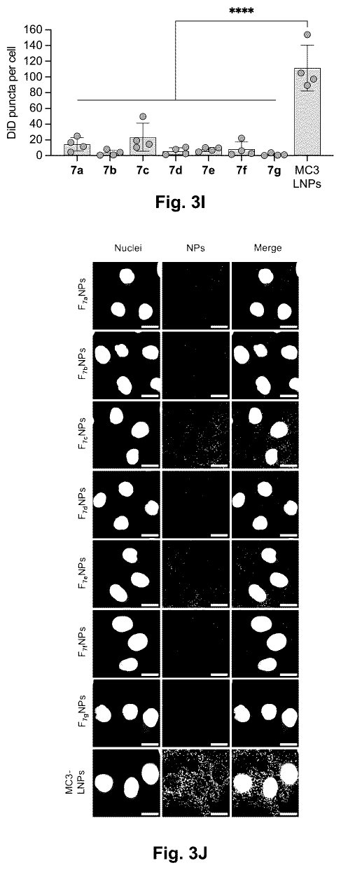

Fulvestrant, a non-ionizable colloid forming drug, was previously chemically modified to produce 13 analogues (7a-7m in FIG. 2) containing ionizable amine groups with pKₐ values ranging from 5.1 to 8.1¹⁰ (FIG. 2). A standard LNP formulation was adapted with ionizable fulvestrant analogues replacing the ionizable lipid (herein termed F_{X}NPs). Specifically, we combined siRNA with each of the ionizable fulvestrant analogs, cholesterol, a phospholipid, and 1,2-dimyristoyl-rac-glycero-3-methoxypolyethylene glycol-2000 (DMG-PEG-2000) (at mole ratios commonly used in LNPs²³ of 50:38.5:10:1.5). Fulvestrant analogues and lipid stabilizing excipients in ethanol were mixed with siRNA in acetate at low pH to allow electrostatic complexation of the cationic ionized drug and anionic RNA phosphates. Microfluidic mixing was employed to achieve uniform sized nanoparticles. Particle surface charge was neutralized by deprotonating non-complexed fulvestrant analogues by buffer exchange into phosphate-buffered saline. Seven analogs (7a-7g) formed relatively small (FIG. 3A) and uniform (FIG. 3B) nanoparticles encapsulating siRNA (F_{X}NPs, where X is one of 7a to 7g) that remained stable for 2 d when formulated with the phospholipid distearylphosphatidylcholine (DSPC). In contrast, the remaining analogs (7h-7m) formed unstable nanoparticles that grew in diameter during storage. Moreover, nanoparticles formulated with 7a-7g were stable in storage for at least 2 years at 4° C. in terms of diameter (FIG. 3C) and polydispersity index (FIG. 3D), except F_{7g}NPs which had a multimodal size distribution after 7 d and FANPs which had a multimodal size distribution after 306 d. The encapsulation (FIG. 3E) and loading (FIG. 3F) efficiencies of siRNA was high for all ionizable analog formulations but low for unmodified fulvestrant (Table 2), confirming the importance of electrostatic interactions for siRNA complexation. A zeta potential between +10 mV and −10 mV is typically considered close to neutral;²⁴ F_{X}NPs were close to neutral or slightly negative (FIG. 3G), and zeta potential showed a positive correlation with ionizable drug pKₐ (FIG. 3H). Cellular uptake in SKOV3 cells of F_{X}NPs with DSPC with a fluorescent tracer dye, DiD, was evaluated by fluorescence microscopy and was significantly lower than a conventional LNP formulation employing the ionizable lipid MC3 (FIG. 3I, representative images in FIG. 3J). Therefore, formulations were tested with alternative phospholipids to improve cellular uptake.

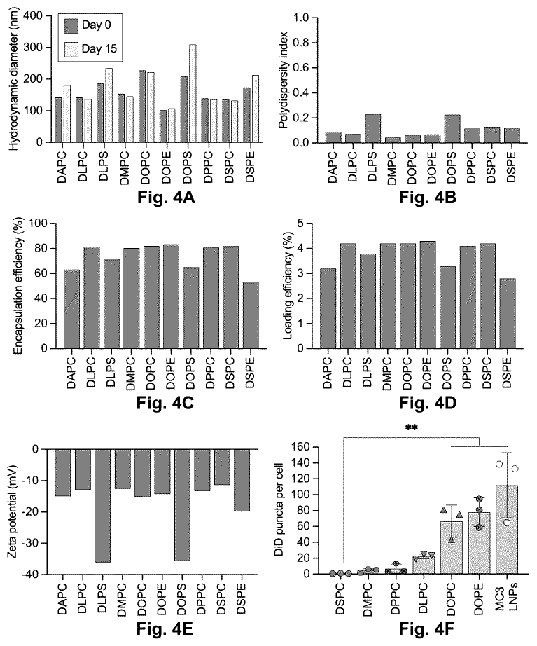

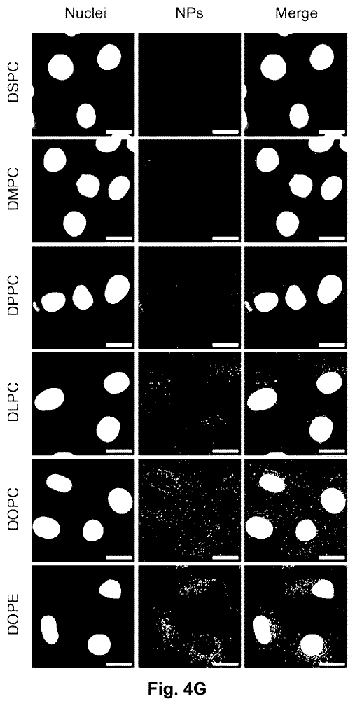

F_{7d}NPs were formulated with an equivalent formulation as above, except DSPC was replaced with one of the following phospholipids: dilauroylphosphatidylcholine (DLPC), dipalmitoylphosphatidylcholine (DPPC), diarachidoylphosphatidylcholine (DAPC), dimyristoylphosphatidylcholine (DMPC), diolcoylphosphatidylcholine (DOPC), diolcoylphosphoethanolamine (DOPE), distearylphosphoethanolamine (DSPE), dilauroylphosphatidylserine (DLPS), or dioleoylphosphatidylserine (DOPS). Formulations had initial sizes between 104 and 228 nm, which were stable over 15 days of 4° C. storage, except for those with DAPC, DLPS, DOPS or DSPE (FIG. 4A). Polydispersity indices were low (<0.2), except for formulations with DLPS or DOPS (FIG. 4B). siRNA encapsulation (FIG. 4C) and loading (FIG. 4D) efficiencies were high, but lower for formulations with DLPS or DOPS. Zeta potentials were slightly negative for most formulations and strongly negative for formulations with negatively charged phospholipids DLPS or DOPS (FIG. 4E). Importantly, two phospholipids, DOPC and DOPE, could significantly improve cellular uptake in SKOV3 cells relative to a DSPC-containing formulation, with a similar level of uptake to control MC3-LNPs (FIG. 4F, representative images in FIG. 4G).

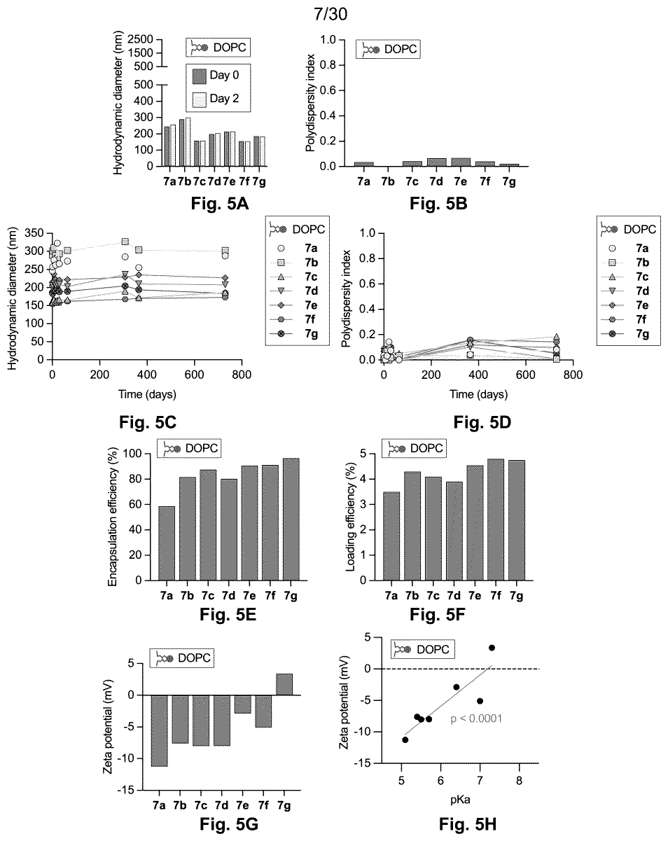

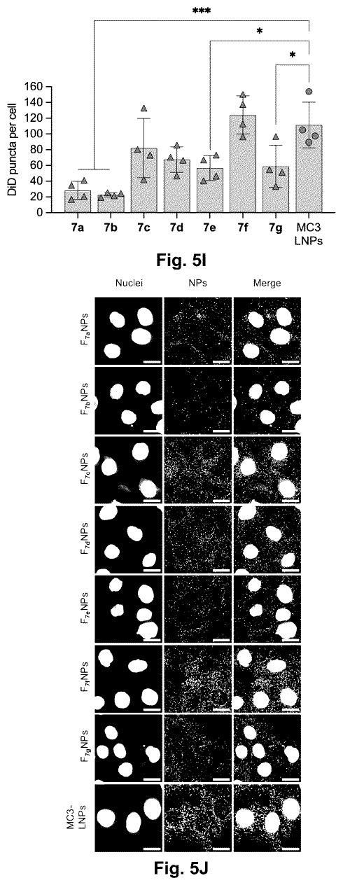

For F_{X}NPs with DOPC, analogs 7a-7g formed relatively small (FIG. 5A) and uniform (FIG. 5B) nanoparticles that remained stable for at least 2 years at 4° C. in terms of diameter (FIG. 5C) and polydispersity index (FIG. 5D), had high encapsulation (FIG. 5E) and loading (FIG. 5F) efficiencies of siRNA, and had zeta potentials that were close to neutral or slightly negative (FIG. 5G) and positively correlated with pKₐ (FIG. 5H). In contrast to F_{X}NPs with DSPC, F_{X}NPs with DOPC demonstrated robust uptake in SKOV3 cells for most fulvestrant analogs (FIG. 5I, representative images in FIG. 5J). F₇ₐNPs and F_{7b}NPs were not meaningfully taken up by cells, regardless of the phospholipid employed, so they were excluded from further study. This was unexpected but may be a result of the relatively large size (>200 nm) of F₇ₐNPs and F_{7b}NPs when formulated with DOPC. DOPC-mediated improvement in cellular uptake may be due to increased adsorption of apolipoprotein E (ApoE), the importance of which was shown quantitatively (FIG. 6) and with representative images (FIG. 7), and is consistent with other reports using standard lipid nanoparticles²⁵.

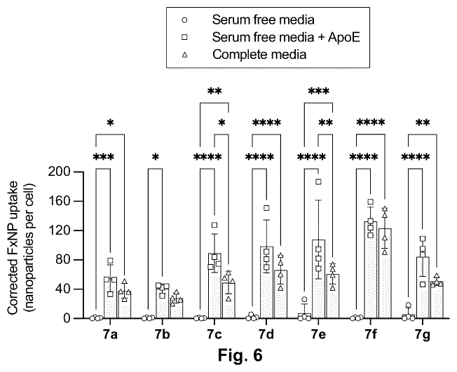

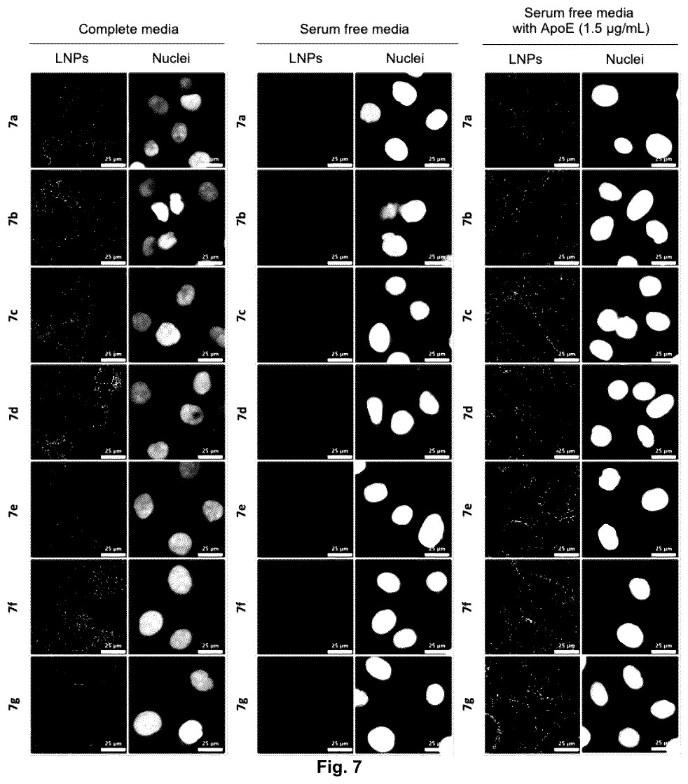

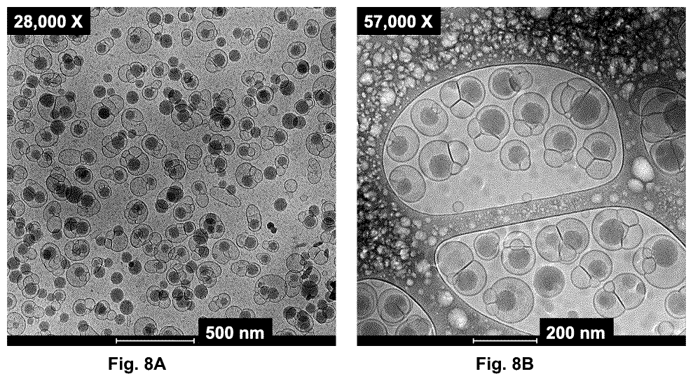

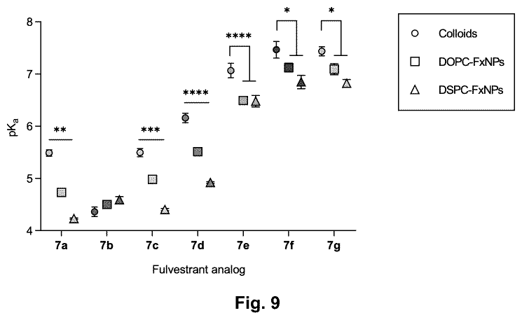

Cryogenic transmission electron microscopy (cryo-TEM) imaging of F_{7f}NPs encapsulating siRNA revealed a morphology with an electron dense spherical core surrounded by bilayer enclosed compartments (FIG. 8). Interestingly, the pKₐ values of the fulvestrant analogs were lower in F_{X}NP formulations than in the corresponding colloidal formulations (FIG. 9), which is consistent with previous findings that excipients impact the pKₐ values of ionizable lipids²⁶.

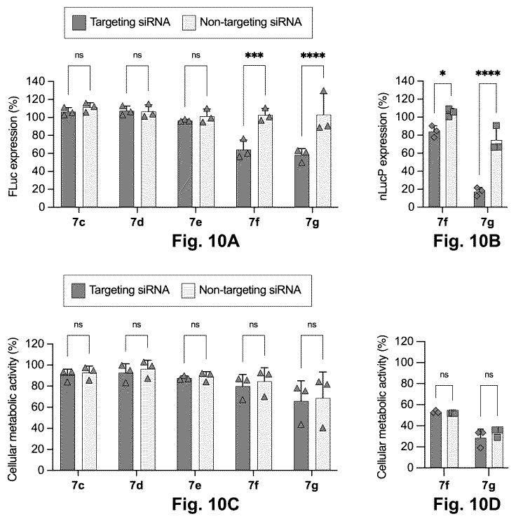

The remaining F_{X}NP formulations (7c-7g) were evaluated for reporter gene knockdown in two cell lines. SKOV3 cells that express firefly luciferase (FLuc) and Vero E6 monkey kidney epithelial cells that express nanoluciferase (nLuc) were treated with formulations encapsulating siRNA targeting either Fluc (siFLuc) or nLuc (sinLuc), respectively, for 24 h. In both cell lines, targeting siRNA produced a significant reduction in model gene expression relative to non-targeting siRNA for F_{7f}NPs and F_{7g}NPs (FLuc in FIG. 10A; and nLuc in FIG. 10B). Importantly, there was no significant difference in cell metabolic activity between equivalent targeting and non-targeting siFLuc and sinLuc formulations (SKOV3-FLuc in FIG. 10C and Vero-nLuc in FIG. 10D), demonstrating that gene knockdown was independent of cell viability. This study illustrates that endosomal and lysosomal disruption by certain ionizable fulvestrant analogs mediates delivery into the cytoplasm.

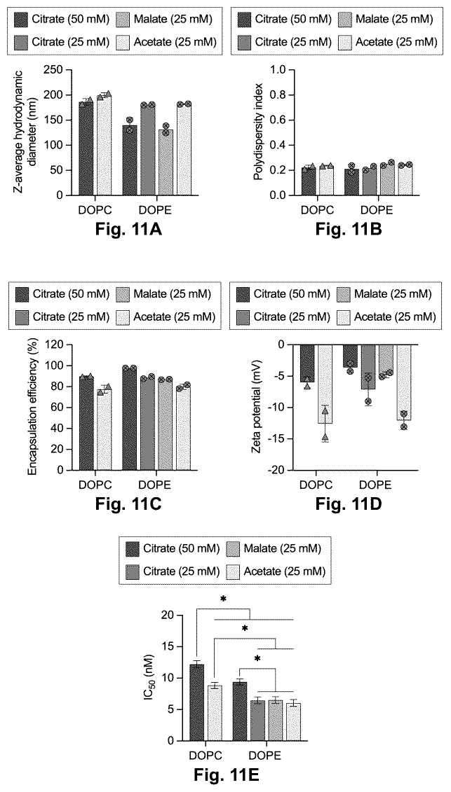

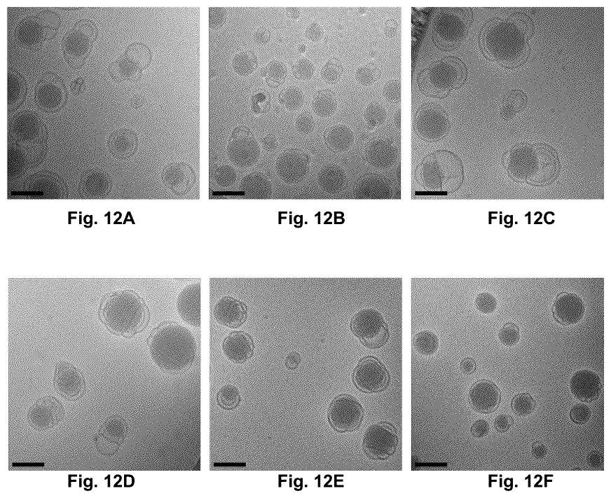

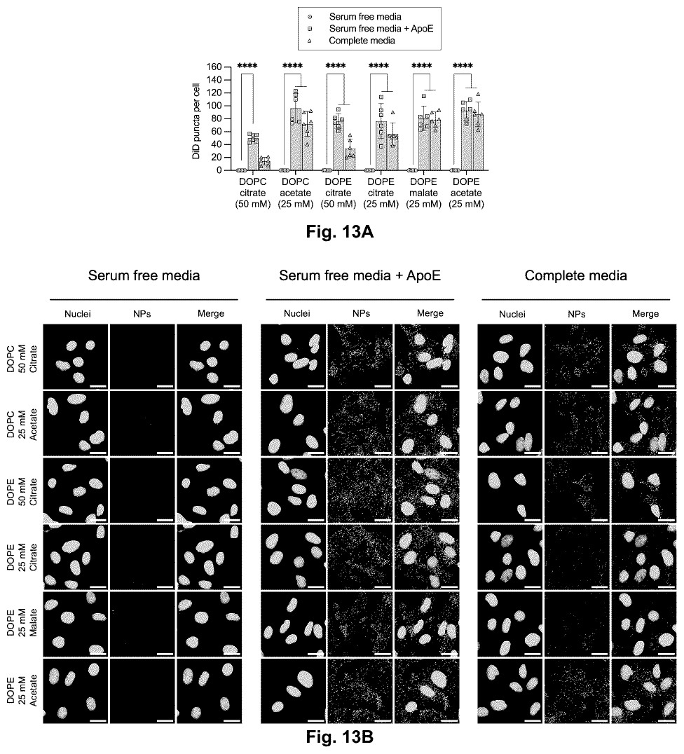

F_{7g}NPs, which exhibited the greatest potency with DOPC, were further optimized by testing an additional phospholipid (DOPE) and comparing 4 different pH 4.0 formulation buffers (50 mM citrate, 25 mM citrate, 25 mM malate, and 25 mM acetate). These formulations were characterized in terms of hydrodynamic diameter (FIG. 11A), polydispersity index (FIG. 11B), siRNA encapsulation efficiency (FIG. 11C), zeta potential (FIG. 11D), and luciferase knockdown potency in SKOV3-FLuc cells (FIG. 11E). The most potent particles were those with DOPE and formulated in one of the 25 mM buffers. Malate in particular resulted in the smallest size, with low polydispersity, near-neutral surface charge and encapsulation efficiency greater than 80%. Therefore, F_{7g}NPs with DOPE and 25 mM malate were chosen as the optimal formulation. We observed that the choice of phospholipid and formulation buffer influenced particle morphology by cryo-TEM, with an increased buffer strength resulting in a larger particle core and more branched aqueous compartments (FIG. 12). It was found that all F_{7g}NP formulations tested were endocytosed in an ApoE (FIG. 13A, representative images in FIG. 13B) and dynamin (FIG. 14A, representative images in FIG. 14B) dependent manner in SKOV3 cells.

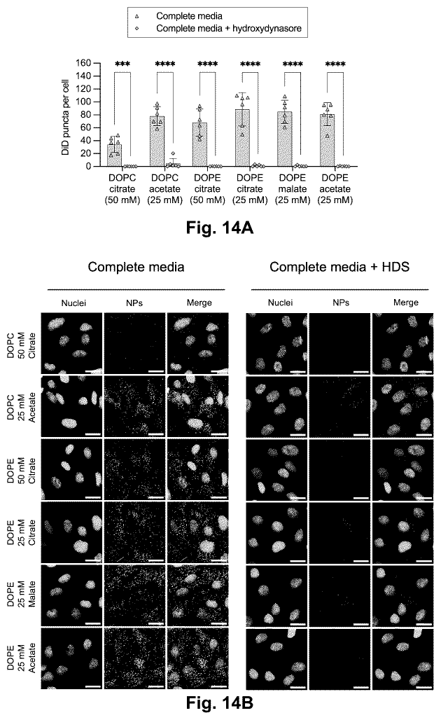

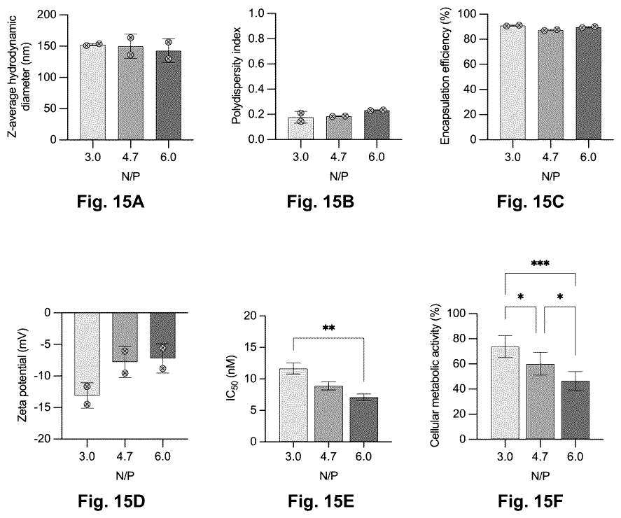

We also evaluated the impact of changing the amount of siRNA loaded in particles, as measured by the ratio of ionizable drug amines (N) to siRNA phosphates (P; N/P ratio). We found that F_{7g}NPs with N/P ratios of 3.0, 4.7, or 6.0 had similar hydrodynamic diameter (FIG. 15A), polydispersity index (FIG. 15B), encapsulation efficiency (FIG. 15C) and zeta potential (FIG. 15D). However, an increase in N/P increased luciferase knockdown potency (Fig. S15E) but also increased cytotoxicity (Fig. S15F). This data highlights a tradeoff where increasing potency requires more delivery material per siRNA which can increase toxicity.

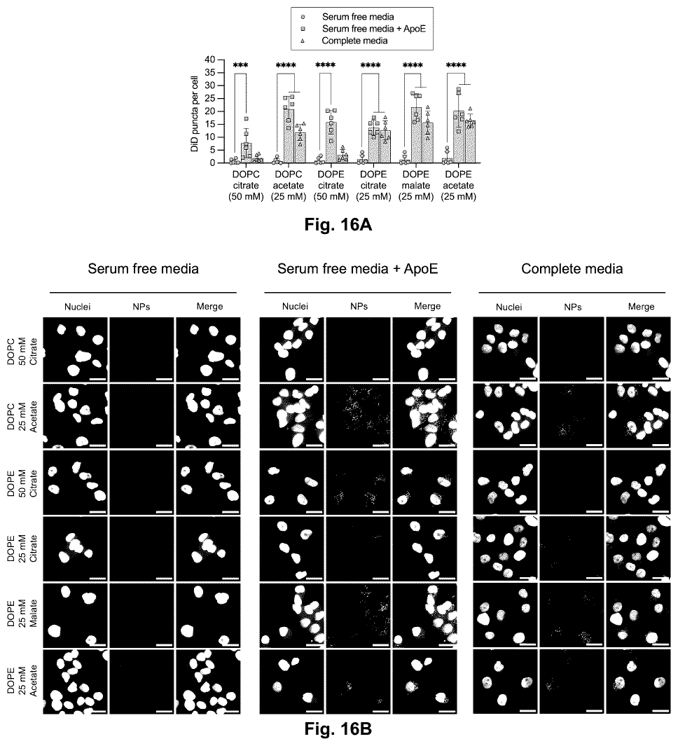

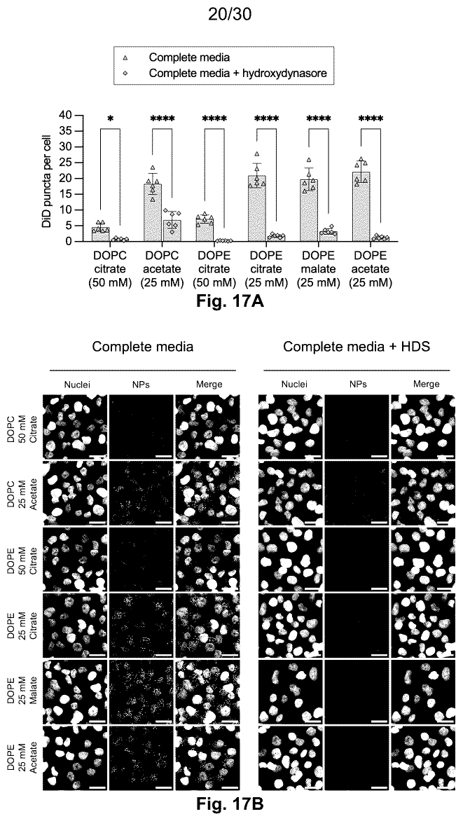

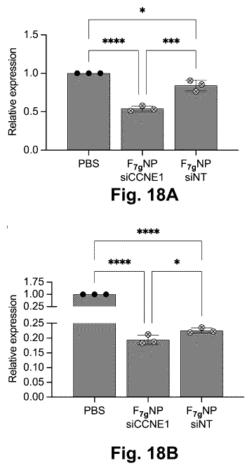

To demonstrate the utility of FNPs in a therapeutically relevant context, particles were loaded with siRNA targeting cyclin E1 (siCCNE1), which is overexpressed in drug-resistant breast cancer cells.²⁷⁻²⁹ T47D breast cancer cells with resistance to palbociclib (T47D-R) that overexpress cyclin E1 were used to test this approach. F_{7g}NPs were endocytosed in T47D-R cells in an ApoE (FIG. 16A, representative images in FIG. 16B) and dynamin (FIG. 17A, representative images in FIG. 17B) dependent manner. We found that F_{7g}NPs with encapsulated siCCNE1 could significantly reduce CCNE1 (the gene encoding cyclin E1) expression relative to F_{7g}NPs with non-targeting siRNA (siNT; FIG. 18A). Additionally, F_{7g}NPs with either siRNA delivered active fulvestrant, which was shown by reduction in GREB1 expression, a gene downstream of fulvestrant's action on the estrogen receptor (FIG. 18B).

### mRNA Encapsulation and Delivery by Ionizable Fulvestrant Analogs

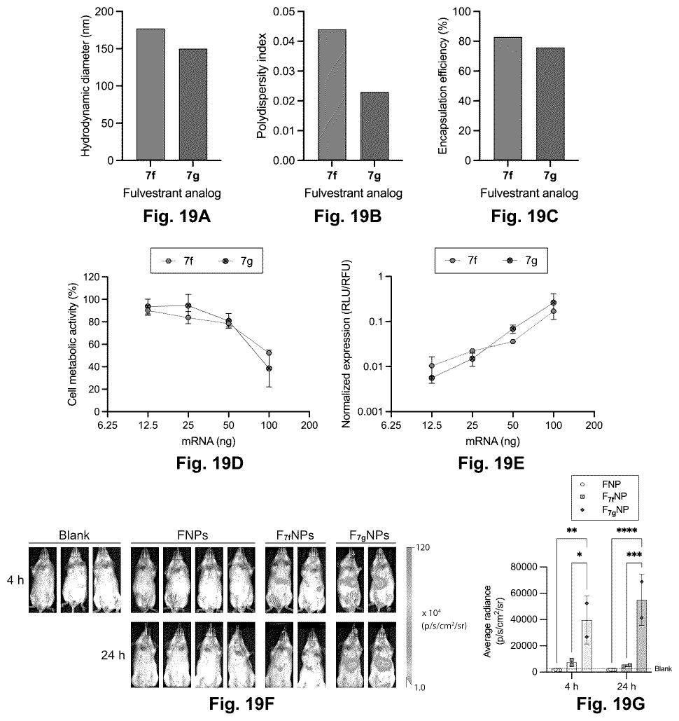

Using the same methodology and composition as employed for siRNA, F_{7f}NPs and F_{7g}NPs were formulated with DOPC and encapsulating mRNA encoding firefly luciferase (mFLuc). This resulted in particles with small (<200 nm; FIG. 19A) and uniform (PDI<0.2; FIG. 19B) diameters, and high encapsulation efficiency (>75%; FIG. 19C). F_{7f}NPs and F_{7g}NPs encapsulating mFLuc resulted in dose dependent expression of luciferase in HepG2 cells (FIGS. 19D, 19E). F_{7f}NPs and F_{7g}NPs encapsulating mFLuc were injected in the tail vein of mice. After 4 and 24 h, luciferin (a luminescent substrate for luciferase) was administered intraperitoneally. We observed bioluminescence in the mouse liver for both formulations, but an equivalent formulation employing unmodified fulvestrant did not show bioluminescence above the baseline (images in FIG. 19F, quantification in FIG. 19G).

### RNA Co-Formulation With Additional Ionizable Drugs

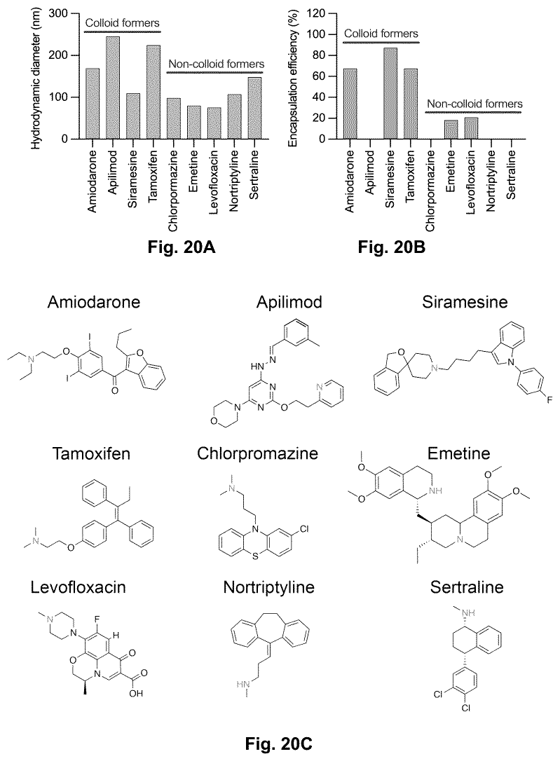

Having demonstrated proof-of-concept siRNA and mRNA delivery with ionizable fulvestrant analogs, we were interested in testing this approach with natively ionizable drugs. Formulations encapsulating siRNA were prepared using the same method as F_{X}NPs except with different ionizable drugs instead of fulvestrant analogs (FIG. 20). Of the 9 drugs tested, 6 resulted in siRNA encapsulation efficiency of <20%, 2 showed moderate encapsulation efficiency (˜60%) and 1 (siramesine) had a high degree of encapsulation efficiency (˜85%). 5 drugs tested were not colloid formers (chlorpromazine, emetine, levofloxacin, nortriptyline, and sertraline) and did not encapsulate siRNA in nanoparticles or encapsulated siRNA at low efficiency, as expected. Among colloid formers, there were differing degrees of siRNA encapsulation.

For drugs with poor siRNA encapsulation, chemical modification can be employed to improve the co-formulation (FIG. 21). This approach was demonstrated with fulvestrant above and can also be applied to sorafenib and emetine. Sorafenib is not natively ionizable, but addition of an ionizable moiety improves siRNA encapsulation (FIGS. 21A, 21B). Emetine is ionizable but does not readily form colloidal drug aggregates. Modification of emetine with a hydrophobic tail to increase hydrophobicity allows colloid formation and improves siRNA encapsulation (FIGS. 21C, 21D). Chemical structures of these drugs are shown in FIG. 21E.

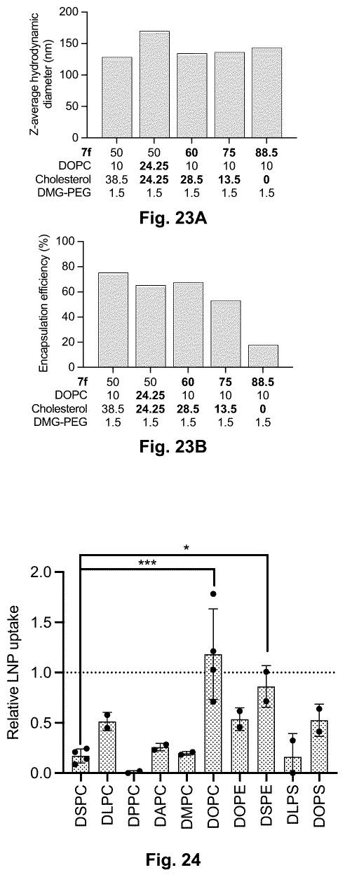

Thus far, we have used compositions based on standard LNP formulations with the ionizable lipid replaced by an ionizable drug. We were interested in understanding the importance of different stabilizing lipid components. As a model drug, we used siramesine, which demonstrated high siRNA encapsulation (˜95%) and small particle size (˜100 nm). In conventional LNP systems both cholesterol and phospholipids are found in a bilayer that surrounds the LNP and stabilizes the structure³⁰. Specifically, the phospholipid is responsible for forming the bilayer structure and cholesterol stabilizes this structure by influencing membrane fluidity³¹. For siramesine nanoparticles, cholesterol was a critical component of the formulation, as its removal result in a 4-fold increase in particle size and ˜70% loss of encapsulated siRNA (FIG. 22). Similarly, removal of cholesterol significantly reduced siRNA encapsulation for F_{7f}NPs (FIG. 23). The choice of phospholipid stabilizer also significantly impacts the behaviour of siramesine nanoparticles. Siramesine nanoparticle formulations were prepared with 10 different phospholipids, and cellular uptake was investigated (FIG. 24). DOPC and DSPE were found to significantly improve uptake of siramesine nanoparticles in cancer cells relative to DSPC-based nanoparticles. This was similar to results observed with fulvestrant analog formulations where DOPC and DOPE improved uptake.

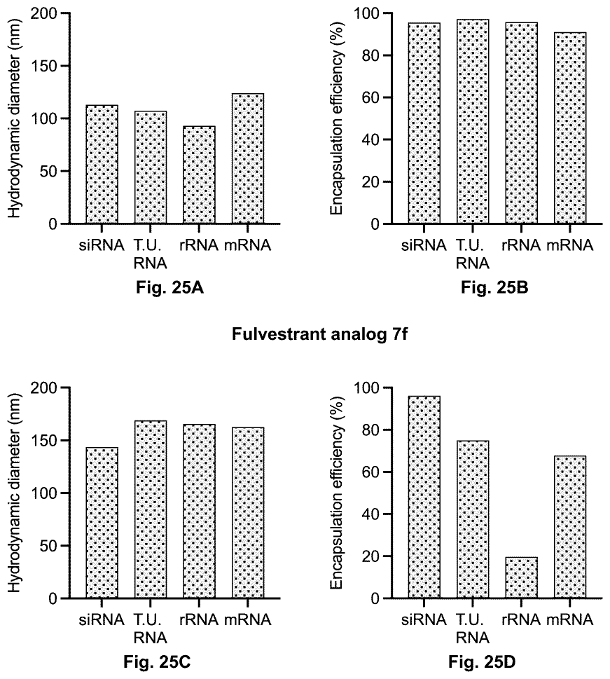

The co-formulation and co-delivery strategy of the present disclosure holds promise for multiple types of RNA therapeutics. Siramesine and fulvestrant analog 7f were shown to form nanoparticles encapsulating a range of RNA cargos of varying molecular weight (FIG. 25).

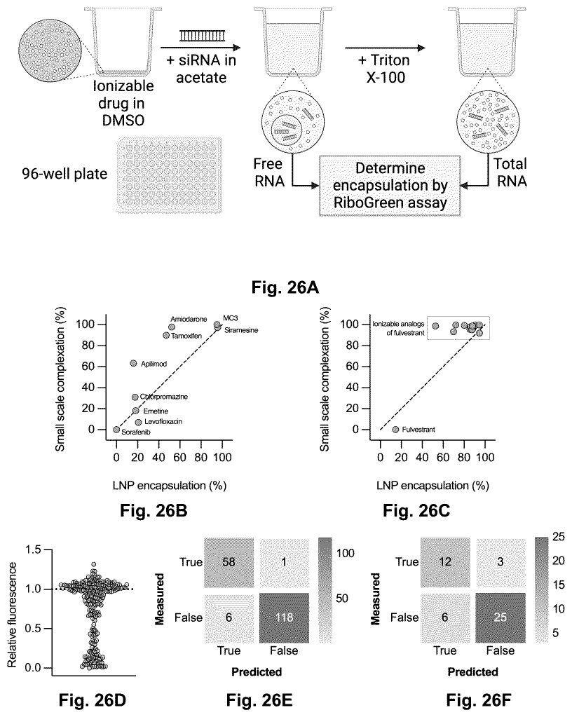

In addition to the drugs described thus far, we aimed to determine what additional drugs can be co-formulated with siRNA using this method. Formulation of ionizable drug nanoparticles with lipid excipients requires a minimum batch size and multiple steps such that testing many drugs would incur significant cost and resources. Therefore, we developed a small-scale screening assay to identify drugs that can complex siRNA (FIG. 26A) and therefore might be useful in a nanoparticle co-formulation. Drug stock solutions at 10 mM in DMSO were added to wells in a 96 well plate and combined with siRNA in pH 4.0 acetate buffer (25 mM). RiboGreen dye was used to detect non-complexed siRNA to estimate complexation efficiency. Addition of the surfactant Triton X-100 was used to dissolve complexes and measure total siRNA in each well. We found that complexation efficiency measured in this small-scale assay was well correlated with siRNA encapsulation efficiency in LNP formulations for multiple drugs (FIG. 26B) and fulvestrant analogs (FIG. 26C). A screening library was chosen from the online database DrugBank® using the following criteria: hydrophobic (log P>3), ionizable (4<pKₐ<11), cationic at low pH (physiological charge≥0) and commercially available (cross-referenced screening library from MedChemExpress). 215 drugs were screened using the small-scale screening method, which allowed the identification of 55 siRNA complexing compounds (FIG. 26D). This data was combined with our prior knowledge of siRNA complexation by ionizable fulvestrant analogs and other drugs for a total of 229 compounds. A random forest model was fit using 5666 molecular descriptors generated for each compound from the alvaDesc descriptor set. The 229 drugs were split into a training set (80%) and testing set (20%), where the training set was used to fit the model and the testing set was used to assess performance. This model was able to predict siRNA complexation by ionizable drugs with balanced accuracies of 96.7% and 80.3% for the training set (FIG. 26E) and testing set (FIG. 26F), respectively. This model can be used to predict the siRNA complexation ability of drugs not tested in this drug screening assay.

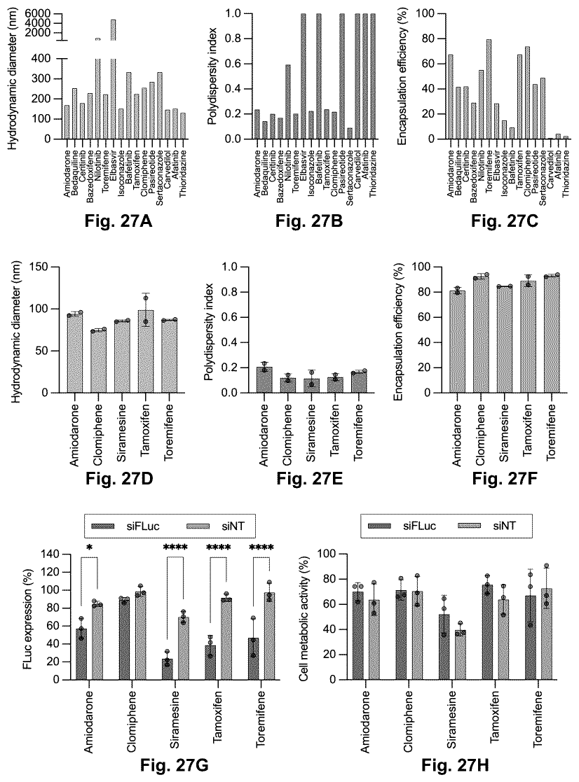

16 drugs that were able to complex siRNA in the screening assay were evaluated in nanoparticle formulations. We began with the lipid composition that was used in initial F_{X}NP formulations that employs DSPC. This produced particles with a range of hydrodynamic diameters (FIG. 27A), where 2 formulations form micron-sized aggregates. The majority of nanoparticles had low PDI, but some had a multimodal distribution (FIG. 27B). A range of siRNA encapsulation efficiencies were observed (FIG. 27C). Although all of these drugs complex siRNA in the small-scale screening assay, they formed particles of varying quality. Some drugs may have improved formulation properties through further optimization of the composition and formulation process.

Using the 5 drugs with the highest siRNA encapsulation efficiency, we prepared nanoparticle formulations using a composition that maximized potency for F_{X}NPs, employing DOPE and formulated in 25 mM acetate. This produced particles with small size (FIG. 27D), low PDI (FIG. 27E) and high siRNA encapsulation efficiency (FIG. 27F). Encapsulation efficiencies were improved compared to DSPC formulations (FIG. 27C), highlighting the importance of the tailoring the composition for a given ionizable drug. For 4 of these drugs, nanoparticle formulations with siFLuc could significantly reduce FLuc expression in SKOV3-FLuc cells compared to those with siNT (FIG. 27G) and this effect was independent of cell metabolic activity (FIG. 27H). Overall, this data demonstrates that the small-scale screening method allowed identification of additional ionizable drugs that can effectively deliver siRNA in a nanoparticle co-formulation.

In addition to identification of drugs through this screening method, the information gained from this assay allowed development of a computational model to predict siRNA complexation by drugs that were not tested. Of the 11,834 small molecule drugs listed on the DrugBank database, our model predicts 545 of these can complex siRNA. A formulation employing one of these drugs, netarsudil, is described in Example 2.

In the present disclosure we demonstrated the ability of certain ionizable drugs to complex RNA in drug-rich co-formulations. While small molecule drugs can be delivered in liposomes, the drug loading is typically limited to less than 10% by mass³², which is significantly less than that of our colloids, which are typically 50-80% drug by mass. Combining high doses of both drugs in a spatiotemporally controlled manner will enable synergistic therapies. For example, an RNA drug can target an undruggable pathway exploited by drug-resistant cancer. As such, the present disclosure encompasses the use of the co-formulation of ionizable drugs and nucleic acid therapeutic, in the treatment of a range of conditions that are the target of the ionizable drug and the nucleic acid therapeutic.

Here, we use a composition based on lipid nanoparticles (LNPs), which are a leading technology for siRNA delivery and employ ionizable lipids for nucleic acid complexation and endosomal escape³³. LNPs contain an oily lipid core that is similar to the core of colloidal drug aggregates. Previous work has shown that many ionizable drugs can induce endosome escape of siRNA delivered via nanocarriers when administered separately to cells^{34,35}. Notably, this effect requires the presence of an endosome escape enhancing drug and siRNA in the same intracellular compartment—conditions that would be achieved by encapsulating siRNA in ionizable colloidal drug aggregates.³³

This work demonstrated the feasibility of this co-delivery approach, but further studies should be undertaken to improve the potency of RNA and small molecule drug delivery. The chosen lipid formulation was adapted directly from a clinically used LNP formulation designed for ionizable lipids. Some work was done to explore the impact of changing the ratio of lipid excipients, including removal of cholesterol. Optimization of the lipid composition could potentially improve the RNA delivery potency. The ratio of ionizable drug to RNA also would need to be selected based on a determined synergistic ratio. In the present work, delivery of siRNA targeting or mRNA encoding a reporter gene was demonstrated.

We have demonstrated the ability of ionizable drugs (including ionizable analogs of non-ionizable drugs) to encapsulate and deliver RNA therapeutics. This allows the delivery of high doses of both drugs into cells. Ionizable fulvestrant analogs were shown to deliver functional siRNA in cancer cells and mRNA in the liver of mice. Further, additional ionizable drugs were able to be co-formulated with RNA. Ultimately, our platform technology will enable synergistic treatments that are currently out of reach.

### Example 2

### Incorporation of Ionizable Lipids in Co-Formulations of Ionizable Drugs and siRNA

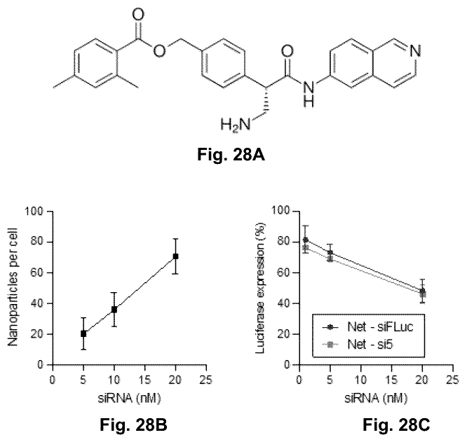

While we showed that replacement of ionizable lipids in standard LNP formulations with ionizable drugs facilitated effective siRNA delivery, inclusion of ionizable lipids can allow for endosomal escape where an ionizable drug efficiently encapsulates siRNA in nanoparticles but fails to result in intracellular delivery or where delivery potency is lower than desired. One such drug is netarsudil (FIG. 28A), a rho-kinase inhibitor used in glaucoma treatment. Formulations encapsulating siRNA were prepared using the same method as F_{X}NPs but using netarsudil instead of fulvestrant analogs. DOPC was employed as a stabilizing phospholipid, as it was shown to facilitate cellular uptake with the other ionizable drugs tested. This resulted in nanoparticles with a hydrodynamic diameter of 138.0 nm, a PDI of 0.184, an encapsulation efficiency of 96.0% and a loading efficiency of 4.99%. This formulation could be efficiently endocytosed into SKOV3-FLuc cells (FIG. 28B) but did not result in specific knockdown of FLuc (FIG. 28C).

We found that a significant fraction of netarusdil was lost during nanoparticle formulation. Netarsudil has 2 ionizable amines, with previously determined pKₐ values of 5.43 and 7.9136 Since the pH of the buffer used for neutralization (PBS; pH 7.4) is lower than one of the pKₐ values, we hypothesized that a significant fraction of the drug was ionized, resulting in increased solubility and loss during centrifugal filtration following neutralization. Therefore, subsequent formulations were neutralized using a buffer with increased pH (Tris-buffered saline; TBS; pH 8.5), which improved netarsudil recovery.

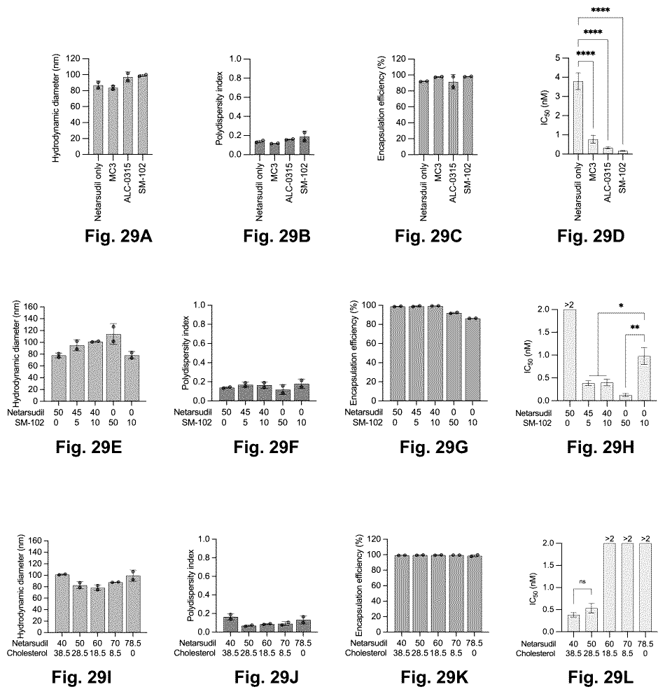

Netarsudil formulations neutralized in TBS had a small (FIG. 29A) and uniform (FIG. 29B) size, with efficient encapsulation of siRNA (FIG. 29C). The IC₅₀ for luciferase knockdown in SKOV3-FLuc cells for formulations with FLuc was 3.8 nM (FIG. 29D). In order to improve the knockdown efficiency of netarsudil nanoparticles, a small amount (10 mol %) of ionizable lipid was added. Three ionizable lipids were tested that efficiently deliver RNA in clinically used formulations: MC3 (Onpattro®), ALC-0315 (BioNTech Pfizer SARS-COV-2 vaccine) and SM-102 (Moderna SARS-COV-2 vaccine). Netarsudil formulations incorporating these ionizable lipids had similarly small hydrodynamic diameters (FIG. 29A), low PDIs (FIG. 29B), and high encapsulation efficiencies of siRNA (FIG. 29BC). All three of these formulations with ionizable lipid significantly improved luciferase knockdown potency relative netarsudil alone when loaded with siFLuc (FIG. 29D). A formulation containing SM-102 was the most potent with an IC₅₀ value of 0.16 nM compared to IC₅₀ values of 0.32 nM and 0.77 nM for formulations using ALC-0315 and MC3, respectively. These data demonstrate that incorporation of small amounts of ionizable lipids in ionizable drug formulations can improve siRNA delivery potency.

The impact of adding ionizable lipid to netarsudil formulations was evaluated further by varying the ratio of netarsudil to SM-102, with ratios of 50:0, 45:5, 40:10, 0:50, and 0:10 for netarsudil:SM-102. We found that all formulations had similarly small sizes (FIG. 29E), low PDIs (FIG. 29F) and high siRNA encapsulation efficiencies (FIG. 29G). The range of concentrations tested was narrowed (<2 nM) to allow for a more accurate comparison between groups, so the IC₅₀ of particles with 50:0 netarsudil:SM-102 was out of this range and not determined (reported as >2; FIG. 29H). SM-102 content could be reduced from 10 mol % to 5 mol % without sacrificing potency. Replacing all of the netarsudil with SM-102 (0:50) resulted in non-significant decrease in IC₅₀. When netarsudil was removed and not replaced (0:10), a decrease in potency and loss of encapsulated siRNA was observed, highlighting the importance of netarsudil in these hybrid formulations.

Formulations were also evaluated where cholesterol was reduced and replaced with netarsudil, with ratios of netarsudil:cholesterol of 40:38.5, 50:28.5, 60:18.5, 70:8.5 and 78.5:0. All formulations had similarly small size (FIG. 29I) and low PDI (FIG. 29J). In contrast to previous observations with fulvestrant analogs and siramesine, reduction in cholesterol didn't cause a loss of siRNA encapsulation (all >98%), even with complete removal of cholesterol (FIG. 29K). However, reduction in cholesterol caused a significant loss in luciferase knockdown potency (FIG. 29L). This data demonstrates that cholesterol is still important in these hybrid netarsudil/SM-102 formulations and highlights the importance of tailoring the composition for a given ionizable drug.

TABLE 2
Physiochemical characterization of FxNP formulations.

| A | B | C | D | E | F | G | H | I | J | K |
|---|---|---|---|---|---|---|---|---|---|---|
| 1 | FV* | — | siFLuc | DSPC | 204.7 | 0.102 | 14.5 | −19.23 | 52.8 | 0.9 |
| 2 | 7a | 5.1 | siFLuc | DSPC | 149.3 | 0.022 | 69.9 | −12.97 | 56.3 | 3.6 |
| 3 | 7b | 5.4 | siFLuc | DSPC | 141.0 | 0.019 | 85.5 | −12.50 | 55.2 | 4.4 |
| 4 | 7c | 5.5 | siFLuc | DSPC | 136.0 | 0.017 | 89.3 | −10.15 | 55.6 | 4.5 |
| 5 | 7d | 5.7 | siFLuc | DSPC | 154.9 | 0.036 | 88.3 | −11.83 | 56.3 | 4.4 |
| 6 | 7e | 6.4 | siFLuc | DSPC | 132.6 | 0.041 | 90.8 | −11.13 | 55.7 | 4.6 |
| 7 | 7f | 7.0 | siFLuc | DSPC | 147.8 | 0.075 | 94.8 | −10.01 | 54.2 | 4.9 |
| 8 | 7g | 7.3 | siFLuc | DSPC | 170.8 | 0.076 | 94.7 | −4.92 | 56.0 | 4.7 |
| 9 | 7h | 7.3 | siFLuc | DSPC | 160.5 | 0.183 | 86.4 | −7.54 | 55.3 | 4.4 |
| 10 | 7i | 7.4 | siFLuc | DSPC | 338.6 | 0.218 | 52.8 | −3.98 | 57.2 | 2.7 |
| 11 | 7j | 7.6 | siFLuc | DSPC | 337.1 | 0.225 | 72.0 | −3.56 | 56.2 | 3.7 |
| 12 | 7k | 7.8 | siFLuc | DSPC | 426.9 | 0.060 | 80.2 | −8.53 | 55.1 | 4.2 |
| 13 | 7l^{†} | — | siFLuc | DSPC | 539.2 | Multimodal | 87.6 | −2.05 | 54.9 | 4.5 |
| 14 | 7m | 8.1 | siFLuc | DSPC | 447.8 | Multimodal | 88.1 | −6.62 | 53.4 | 4.7 |
| 15 | 7a | 5.1 | siFLuc | DOPC | 245.9 | 0.035 | 58.7 | −11.27 | 56.7 | 3.0 |
| 16 | 7b | 5.4 | siFLuc | DOPC | 289.8 | 0.003 | 81.7 | −7.61 | 55.4 | 4.2 |
| 17 | 7c | 5.5 | siFLuc | DOPC | 159.4 | 0.043 | 87.5 | −8.02 | 55.7 | 4.4 |
| 18 | 7d | 5.7 | siFLuc | DOPC | 199.0 | 0.066 | 80.3 | −8.00 | 56.5 | 4.0 |
| 19 | 7e | 6.4 | siFLuc | DOPC | 214.2 | 0.068 | 90.8 | −2.88 | 55.7 | 4.6 |
| 20 | 7f | 7.0 | siFLuc | DOPC | 155.5 | 0.041 | 91.3 | −5.09 | 54.3 | 4.8 |
| 21 | 7g | 7.3 | siFLuc | DOPC | 186.2 | 0.022 | 96.6 | 3.39 | 56.0 | 4.8 |
| 22 | 7a | 5.1 | sinLuc | DOPC | 243.5 | 0.014 | 79.5 | −11.80 | 56.1 | 4.0 |
| 23 | 7b | 5.4 | sinLuc | DOPC | 296.2 | 0.012 | 84.9 | −11.53 | 55.3 | 4.4 |
| 24 | 7c | 5.5 | sinLuc | DOPC | 180.1 | 0.072 | 73.7 | −10.97 | 56.1 | 3.8 |
| 25 | 7d | 5.7 | sinLuc | DOPC | 224.8 | 0.093 | 76.4 | −14.27 | 56.6 | 3.8 |
| 26 | 7e | 6.4 | sinLuc | DOPC | 215.1 | 0.066 | 89.7 | −7.23 | 55.7 | 4.5 |
| 27 | 7f | 7.0 | sinLuc | DOPC | 161.0 | 0.039 | 92.4 | −3.97 | 54.3 | 4.8 |
| 28 | 7g | 7.3 | sinLuc | DOPC | 181.8 | 0.042 | 94.9 | 0.01 | 56.0 | 4.7 |

*Non-ionizable parent drug
^{†}Permanently ionized fulvestrant analogue
Abbreviations of first row of Table 2:
A: Formulation ID;
B: Ionizable compound;
C: pKa;
D: siRNA;
E: Phospholipid;
F: Hydrodynamic diameter (nm);
G: PDI;
H: Encapsulation efficiency (%);
I: Zeta potential (mV);
J: Small molecule drug mass %;
K: siRNA mass %

### REFERENCES

1. Saraswathy, M. & Gong, S. Recent developments in the co-delivery of siRNA and small molecule anticancer drugs for cancer treatment. Materials Today 17, 298-306 (2014).
2. Wu, S. Y., Lopez-Berestein, G., Calin, G. A. & Sood, A. K. RNAi Therapies: Drugging the Undruggable. Science Translational Medicine 6, 240ps7-240ps7 (2014).
3. Ickenstein, L. M. & Garidel, P. Lipid-based nanoparticle formulations for small molecules and RNA drugs. Expert Opinion on Drug Delivery 16, 1205-1226 (2019).
4. Younis, M. A., Khalil, I. A., Elewa, Y. H. A., Kon, Y. & Harashima, H. Ultra-small lipid nanoparticles encapsulating sorafenib and midkine-siRNA selectively-eradicate sorafenib-resistant hepatocellular carcinoma in vivo. Journal of Controlled Release 331, 335-349 (2021).
5. Chen, S. et al. Dexamethasone prodrugs as potent suppressors of the immunostimulatory effects of lipid nanoparticle formulations of nucleic acids. J Control Release 286, 46-54 (2018).
6. Ganesh, A. N., Donders, E. N., Shoichet, B. K. & Shoichet, M. S. Colloidal aggregation: From screening nuisance to formulation nuance. Nano Today 19, 188-200 (2018).
7. Ganesh, A. N. et al. Leveraging Colloidal Aggregation for Drug-Rich Nanoparticle Formulations. Mol. Pharmaceutics 14, 1852-1860 (2017).
8. Ganesh, A. N., Mclaughlin, C. K., Duan, D., Shoichet, B. K. & Shoichet, M. S. A New Spin on Antibody-Drug Conjugates: Trastuzumab-Fulvestrant Colloidal Drug Aggregates Target HER2-Positive Cells. ACS Appl. Mater. Interfaces 9, 12195-12202 (2017).
9. Donders, E. N. et al. Triggered Release Enhances the Cytotoxicity of Stable Colloidal Drug Aggregates. ACS Chem. Biol. 14, 1507-1514 (2019).
10. Donders, E. N. et al. Synthetic Ionizable Colloidal Drug Aggregates Enable Endosomal Disruption. Advanced Science 2300311 (2023) doi: 10.1002/advs.202300311.
11. Zhigaltsev, I. V., Tam, Y. Y. C., Kulkarni, J. A. & Cullis, P. R. Synthesis and Characterization of Hybrid Lipid Nanoparticles Containing Gold Nanoparticles and a Weak Base Drug. Langmuir 38, 7858-7866 (2022).
12. Evers, M. J. W. et al. State-of-the-Art Design and Rapid-Mixing Production Techniques of Lipid Nanoparticles for Nucleic Acid Delivery. Small Methods 2, 1700375 (2018).
13. Walsh, C. et al. Microfluidic-Based Manufacture of siRNA-Lipid Nanoparticles for Therapeutic Applications. in Drug Delivery System (ed. Jain, K. K.) vol. 1141 109-120 (Springer New York, New York, NY, 2014).
14. Whitehead, K. A. et al. Degradable Lipid Nanoparticles with Predictable In Vivo siRNA Delivery Activity. Nat Commun 5, 4277 (2014).
15. Heyes, J., Palmer, L., Bremner, K. & MacLachlan, I. Cationic lipid saturation influences intracellular delivery of encapsulated nucleic acids. Journal of Controlled Release 107, 276-287 (2005).
16. Schindelin, J. et al. Fiji—an Open Source platform for biological image analysis. Nat Methods 9, 10.1038/nmeth.2019 (2012).
17. Kilchrist, K. V. et al. Gal8 Visualization of Endosome Disruption Predicts Carrier-Mediated Biologic Drug Intracellular Bioavailability. ACS Nano (2019) doi: 10.1021/acsnano.8b05482.
18. Ye, R. et al. KCTD12 promotes G1/S transition of breast cancer cell through activating the AKT/FOXO1 signaling. J Clin Lab Anal 34, e23315 (2020).
19. Lei, J. T. et al. Functional Annotation of ESR1 Gene Fusions in Estrogen Receptor-Positive Breast Cancer. Cell Rep 24, 1434-1444.e7 (2018).
20. Kuan, C.-T. et al. MRP3: a molecular target for human glioblastoma multiforme immunotherapy. BMC Cancer 10, 468 (2010).
21. Reker, D. et al. Computationally guided high-throughput design of self-assembling drug nanoparticles. Nat Nanotechnol 16, 725-733 (2021).
22. Sushko, I. et al. Online chemical modeling environment (OCHEM): web platform for data storage, model development and publishing of chemical information. J Comput Aided Mol Des 25, 533-554 (2011).
23. Cullis, P. R. & Hope, M. J. Lipid Nanoparticle Systems for Enabling Gene Therapies. Molecular Therapy 25, 1467-1475 (2017).
24. Clogston, J. D. & Patri, A. K. Zeta Potential Measurement. in Characterization of
Nanoparticles Intended for Drug Delivery (ed. McNeil, S. E.) 63-70 (Humana Press, Totowa, NJ, 2011). doi: 10.1007/978-1-60327-198-1_6.
25. Zhang, R. et al. Helper lipid structure influences protein adsorption and delivery of lipid nanoparticles to spleen and liver. Biomater. Sci. 9, 1449-1463 (2021).
26. Zhang, J., Fan, H., Levorse, D. A. & Crocker, L. S. Ionization Behavior of Amino Lipids for siRNA Delivery: Determination of Ionization Constants, SAR, and the Impact of Lipid pKa on Cationic Lipid-Biomembrane Interactions. Langmuir 27, 1907-1914 (2011).
27. Knudsen, E. S. & Witkiewicz, A. K. The Strange Case of CDK4/6 Inhibitors: Mechanisms, Resistance, and Combination Strategies. Trends in Cancer 3, 39-55 (2017).
28. Turner, N. C. et al. Cyclin E1 Expression and Palbociclib Efficacy in Previously Treated Hormone Receptor-Positive Metastatic Breast Cancer. JCO 37, 1169-1178 (2019).
29. Thu, K., Soria-Bretones, I., Mak, T. & Cescon, D. Targeting the cell cycle in breast cancer: towards the next phase. Cell Cycle 17, 1871-1885 (2018).
30. Kulkarni, J. A., Witzigmann, D., Leung, J., Tam, Y. Y. C. & Cullis, P. R. On the role of helper lipids in lipid nanoparticle formulations of siRNA. Nanoscale 11, 21733-21739 (2019).
31. Hald Albertsen, C. et al. The role of lipid components in lipid nanoparticles for vaccines and gene therapy. Adv Drug Deliv Rev 188, 114416 (2022).
32. Park, K. Facing the Truth about Nanotechnology in Drug Delivery. ACS Nano 7, 7442-7447 (2013).
33. Kulkarni, J. A., Witzigmann, D., Chen, S., Cullis, P. R. & Van Der Meel, R. Lipid Nanoparticle Technology for Clinical Translation of siRNA Therapeutics. Accounts of Chemical Research 52, 2435-2444 (2019).
34. Du Rietz, H., Hedlund, H., Wilhelmson, S., Nordenfelt, P. & Wittrup, A. Imaging small molecule-induced endosomal escape of siRNA. Nature Communications 11, 1-17 (2020).
35. Van de Vyver, T. et al. Cationic Amphiphilic Drugs Boost the Lysosomal Escape of Small Nucleic Acid Therapeutics in a Nanocarrier-Dependent Manner. ACS nano 14, 4774-4791 (2020).
36. Roclanda|European Medicines Agency. https://www.ema.europa.eu/en/medicines/human/EPAR/roclanda.

### LIST OF ABBREVIATIONS

ApoE apolipoprotein E
CCNE1 gene encoding cyclin E1
Cryo-TEM cryogenic transmission electron microscopy
DAPC diarachidoylphosphatidylcholine
DiD 1,1′-dioctadecyl-3,3,3′,3′-tetramethylindodicarbocyanine,4-chlorobenzenesulfonate salt
DLPC dilauroylphosphatidylcholine
DLPS dilauroylphosphatidylserine
DLS dynamic light scattering
DMG-PEG-2000 1,2-dimyristoyl-rac-glycero-3-methoxypolyethylene glycol-2000
DMPC dimyristoylphosphatidylcholine
DOPC dioleoylphosphatidylcholine
DOPS dioleoylphosphatidylserine
DOPE dioleoylphosphoethanolamine
DPPC dipalmitoylphosphatidylcholine
DSPC distearoylphosphatidylcholine
DSPE distearoylphosphoethanolamine
FLuc firefly luciferase
F_{X}NP fulvestrant analog nanoparticles encapsulating RNA
HBSS Hank's balanced salt solution
LNP lipid nanoparticle
mRNA messenger RNA
nLuc nano luciferase
PBS phosphate buffered saline
PDI polydispersity index
PEG polyethylene glycol
PFA paraformaldehyde
rRNA ribosomal RNA
SEM standard error of the mean
siCCNE1 siRNA targeting CCNE1
siFLuc siRNA targeting FLuc
sinLuc siRNA targeting nLuc
siNT non-targeting siRNA
siRNA small interfering RNA
TBS Tris-buffered saline
T.U. RNA RNA extracted from Torula utilis yeast
ADDIN EN.REFLIST

What is claimed is:

1. A co-formulation comprising:
(i) an ionizable colloid forming active substance;
(ii) a nucleic acid molecule; and
(iii) an amphiphilic molecule.

2. The co-formulation of claim 1, wherein the ionizable colloid forming active substance is an ionizable analog of a non-ionizable molecule.

3. The co-formulation of claim 1, wherein the ionizable colloid forming active substance comprises an analog of an ionizable non-colloid forming active substance modified to form colloidal aggregates.

4. The co-formulation of claim 3, wherein the analog of the ionizable non-colloid forming active substance includes a hydrophobic group to form colloidal aggregates.

5. The co-formulation of claim 4, wherein the hydrophobic group is lauroyl, myristoyl, palmitoyl, stearoyl, oleoyl, linoleoyl, linolenoyl, or arachidonoyl acyl chains.

6. The co-formulation of claim 3, wherein the modification comprises addition of a piperidine, pyrrolidine or imidazole chemical group to the non-colloid forming active substance.

7. The co-formulation of claim 1, wherein the ionizable colloid forming active substance includes ionizable amine groups.

8. The co-formulation of claim 7, wherein the ionizable amine groups have experimentally determined pKₐ values ranging from 4 to 11.

9. The co-formulation of claim 1, wherein the nucleic acid molecule is a natural nucleic acid molecule or a xeno nucleic acid molecule.

10. The co-formulation of claim 1, wherein the nucleic acid molecule is a DNA molecule or RNA molecule.

11. The co-formulation of claim 10, wherein the DNA or RNA are functional DNA or RNA or decoy DNA or RNA.

12. The co-formulation of claim 11, wherein the functional DNA or RNA include aptamers, plasmid, miRNA, mRNA and siRNA.

13. The co-formulation of claim 1, wherein the ionizable colloid-forming active substance is derived from a non-ionizable, non-colloid forming molecule.

14. The co-formulation of claim 1, wherein the amphiphilic molecule is a polyethylene glycol-conjugated lipid (PEG-lipid), poly (carboxybetaine)-conjugated lipid, cholesterol, a cholesterol analogue, a phospholipid, a phospholipid analogue, or combination thereof.

15. The co-formulation of claim 1, wherein the amphiphilic molecule is a polyethylene glycol-conjugated lipid (PEG-lipid), and wherein the PEG-lipid is 1,2-dimyristoyl-rac-glycero-3-methoxypolyethylene glycol-2000.

16. The co-formulation of claim 14, wherein the amphiphilic molecule is a phospholipid, and wherein the phospholipid is distearylphosphatidylcholine (DSPC), dilauroylphosphatidylcholine (DLPC), dipalmitoylphosphatidylcholine (DPPC), diarachidoylphosphatidylcholine (DAPC), dimyristoylphosphatidylcholine (DMPC), dioleoylphosphatidylcholine (DOPC), dioleoylphosphoethanolamine (DOPE), distearylphosphoethanolamine (DSPE), dilauroylphosphatidylserine (DLPS), dioleoylphosphatidylserine (DOPS) or any combination thereof.

17. The co-formulation of claim 1, wherein the co-formulation has a z-average hydrodynamic diameter of about 1000 nm or less, preferably 200 nm or less.

18. The co-formulation of claim 1, wherein the ionizable colloid forming active substance is an ionizable fulvestrant analog, and wherein the ionizable fulvestrant analog comprises fulvestrant having one or more ionizable amine groups.

19. The co-formulation of claim 1, wherein the active substance is one of netarsudil, amiodarone, emetine, lapatinib, levofloxacin, siramesine, an ionizable fulvestrant analog, bedaquiline, certinib, bazedoxifene, nilotinib, toremifene, elbasvir, isoconazole, bafetinib, clomiphene, pasireotide, sertaconazole, carvediol, afatinib, thioridazine, tamoxifen, or a combination thereof.

20. The co-formulation of claim 18, wherein the ionizable fulvestrant analog is combined with another active substance selected from the group consisting of fulvestrant, netarsudil, amiodarone, apilimod, lapatinib, levofloxacin, siramesine, bedaquiline, certinib, bazedoxifene, nilotinib, toremifene, elbasvir, isoconazole, bafetinib, clomiphene, pasireotide, sertaconazole, carvediol, afatinib, thioridazine and tamoxifen.

21. The co-formulation of claim 1, wherein the active substance is a sorafenib analog or an emetine analog.

22. The co-formulation of claim 1, wherein the co-formulation further comprises an ionizable lipid.

23. The co-formulation of claim 1, wherein the co-formulation is free of ionizable lipids.

24. A method of treating a disease or disorder comprising administering the co-formulation of claim 1 to a subject in need, wherein the ionizable colloid-forming active substance and the nucleic acid molecule included in the co-formulation act together in the treatment of the disease or disorder.

25. The method of claim 24, wherein the disease or disorder is cancer.

26. A method of manufacturing the co-formulation of claim 1, wherein the method comprises:
(a) mixing the ionizable colloid forming active substance with the nucleic acid molecule at a pH lower than the pKa value of the ionizable colloid forming active substance to form a particle having the nucleic acid molecule entrapped therein;
(b) neutralizing the particle's surface charge in a buffer having neutral or close to neutral pH or a pH greater than the largest pKₐ value of the ionizable colloid forming active substance; and
(c) adding an amphiphilic molecule to facilitate entrapment of the nucleic acid molecule within the formed colloid nanoparticle and/or to stabilize the formed colloid nanoparticle.

27. An ionizable analog of emetine comprising emetine modified to include a hydrophobic group.
